(19) United States
(12) Patent Application Publication
(10) Pub. No.: US 2026/0139232 A1
(43) Pub. Date: May 21, 2026

(54) METHOD OF PURIFYING FULL RECOMBINANT AAV PARTICLES

(71) Applicant: PASSAGE BIO, INC., Philadelphia, PA (US)

(72) Inventors: Ronald JENKINS, Philadelphia, PA (US); Nripen SINGH, Philadelphia, PA (US); Aaron COLLIER, Philadelphia, PA (US); Supriya KUMAR, Philadelphia, PA (US); Bin LU, Philadelphia, PA (US)

(21) Appl. No.: 19/120,174

(22) PCT Filed: Oct. 6, 2023
(86) PCT No.: PCT/US2023/076186

§ 371 Date: Apr. 10, 2025

Related U.S. Application Data
(60) Provisional application No. 63/378,997, filed on Oct. 10, 2022.

(51) Int. Cl. C12N 7/00; B01D 15/16; B01D 15/36; B01D 15/38

(52) U.S. Cl. CPC C12N 7/00; B01D 15/166; B01D 15/363; B01D 15/3804; C12N 2750/14151

(57) ABSTRACT

Provided herein are methods for purifying recombinant adeno-associated virus (rAAV) particles using chromatography in a large scale. Further provided include the rAAV particles and the pharmaceutical composition prepared by the method.

### 1. CROSS-REFERENCE TO RELATED APPLICATIONS

This application claims benefit of and priority to U.S. Provisional Application No. 63/378,997, filed Oct. 10, 2022, the disclosure of which is incorporated herein by reference in its entirety.

### 2. BACKGROUND

Adeno-associated virus (AAV) is a non-enveloped virus that belongs to the parvovirus family. AAV has a linear single-stranded DNA (ssDNA) genome of approximately 4.7-kilobases (kb), containing three genes, Rep (Replication), Cap (Capsid), and aap (Assembly).

The Rep gene encodes four proteins (Rep78, Rep68, Rep52, and Rep40), which are required for viral genome replication and packaging and the Cap gene encodes viral capsid proteins (VP; VP1/VP2/VP3), which are required for capsid formation, target cell binding and internalization.

Because of the low genetic complexity, AAVs have attracted a significant interest in the field of gene therapy. The low genetic complexity facilitates the cloning, packaging, and delivery of therapeutic gene expression cassettes to target cells. rAAVs lacking viral genes and containing the gene expression cassette with a gene of interest have been demonstrated to be safe and effective gene therapy vehicles that can deliver the gene of interest to the target in vivo. rAAVs have become the dominant form of gene therapy and rAAV-based therapeutics have received regulatory approval in Europe and the United States.

rAAV particles can be produced in packaging host cell cultures by co-expression of helper virus AAV Rep and AAV Cap genes, for replication and packaging. Typically, the host cells are lysed to release rAAV particles and maximize yield of recovered rAAV. However, the cell lysate contains various cellular components such as host cell DNA, host cell proteins, media components, and in some instances, helper virus or helper virus plasmid DNA. Further, not all the rAAV particles produced and released from host cells contain the genomic DNA with the gene of interest (full rAAV particles). A significant portion of the rAAV particles is without DNA (empty rAAV particles) or contains only partial genomes (partially filled rAAV particles). The empty and partially filled rAAV particles are considered impurities as they increase the dose of total AAV administered for efficient transduction. Therefore, rAAV particles collected from the media and/or cell lysate should be further purified to be suitable for therapeutic use.

Currently available methods for purification of rAAV are not scalable and/or not adaptable to good manufacturing practices. rAAV particles purified using cesium chloride gradient ultra-centrifugation as a purification step have been used in some clinical trials, however the purification methods are not readily scalable. Other methods such as heparin-based affinity column chromatography and ion-exchange chromatography have been used for purification of rAAVs. However, unlike density gradient centrifugation, chromatographic methods generally do not achieve separation of empty capsids from full capsids.

Therefore, a scalable purification method that allows enrichment of rAAV full capsids from empty and partial capsids is needed, particularly to meet the surging demands of rAAVs for rAAV-based therapeutics.

### 3. SUMMARY

The present disclosure relates to methods of purifying rAAV particles. The method allows purification and enrichment of full rAAV particles suitable for clinical applications. Also provided are rAAVs particles and the pharmaceutical composition produced by the methods.

In one aspect, the present disclosure provides a method of purifying a recombinant adeno-associated virus (rAAV) particles, the method comprising:
a) providing a feed composition comprising the rAAV particles, wherein the rAAV particles in the feed composition comprise empty and full rAAV particles;
b) contacting the feed composition with a chromatography media for a time-on-media under a condition that allows binding of the rAAV particles to the chromatography media, wherein the time-on-media is at least 0.5 hr;
c) eluting the rAAV particles from the chromatography media; and
d) recovering purified rAAV particles, thereby enriching full rAAV particles.

In some embodiments, the time-on-media is longer than 2 hrs. In some embodiments, the time-on-media is 2 to 24 hours. In some embodiments, the time-on-media is longer than 3 hrs, 4 hrs, 5 hrs, 6 hrs, 7 hrs, 8 hrs, 9 hrs, 10 hrs, 11 hrs, or 12 hrs.

In some embodiments, the step of contacting comprises holding the rAAV particles bound to the chromatography media within a holding buffer for a hold duration, wherein the hold duration is 0.5 hr or longer. In some embodiments, the hold duration is 0.5 to 24 hours. In some embodiments, the hold duration is not more than 3 hours. In some embodiments, the hold duration is 0.5 to 3 hours. In some embodiments, the hold duration is about 3 hours.

In some embodiments, the holding buffer has a pH 9.0 to 11. In some embodiments, the holding buffer has a pH of about pH 10.2.

In some embodiments, the step of contacting comprises loading the rAAV particles onto the chromatography media over a loading duration. In some embodiments, the loading duration is longer than 0.5 hr, 1 hr, 2 hrs, 3 hrs, 4 hrs, 5 hrs, 6 hrs, 12 hrs, or 18 hrs. In some embodiments, the loading duration is 0.5 to 24 hours.

In some embodiments, the loading duration and the hold duration are 0.5 to 24 hours in total. In some embodiments, the loading duration and the hold duration are at least 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, or 23 hrs in total.

In some embodiments, the step of contacting comprises washing the rAAV particles bound to the chromatography media with a washing buffer having pH 9.0 to 11, after loading the feed composition onto the chromatography media but before holding the rAAV particles bound to the chromatography media. In some embodiments, the washing buffer has a pH of between pH 9.5 to 10.5. In some embodiments, the washing buffer has a pH of about pH 10.2. In some embodiments, the washing buffer comprises Bis-Tris propane (BTP) or glycine.

In some embodiments, the feed composition has a pH between 8.0 and 8.9.

In some embodiments, the chromatography media is an anion exchange chromatography media. In some embodiments, the chromatography media is an affinity chromatography media.

In some embodiments, the eluting step is performed with a linear salt gradient. In some embodiments, the linear salt gradient comprises between about 0.001 mM NaCl and about 1000 mM NaCl. In some embodiments, the linear salt gradient comprises between about 0.001 mM NaCl and about 100 mM NaCl. In some embodiments, the eluting step is performed with a step salt gradient. In some embodiments, the step salt gradient comprises between about 7 mM NaCl and about 500 mM NaCl. In some embodiments, the step salt gradient comprises between about 70 mM NaCl and about 100 mM NaCl.

In some embodiments, the loading step is performed by flowing feed composition through the chromatography media at a flow rate of between 0.1 CV/min and 5 CV/min.

In some embodiments, the chromatography media comprises one or more amine functional groups. In some embodiments, the one or more amine functional group is selected from a primary amine, a secondary amine, a tertiary amine, a quaternary amine functional group, or combinations thereof. In some embodiments, the one or more amine functional group comprises a quaternary amine functional group. In some embodiments, the one or more amine functional groups are bound onto a resin, membrane, and/or nanofiber chromatographic media.

In some embodiments, the chromatography media comprises monolith.

In some embodiments, the step of eluting is performed in a buffer having a pH between 9.5 and 10.5.

In some embodiments, the rAAV particles comprise a capsid protein of an AAV selected from AAV1, AAV2, AAV3, AAV4, AAV5. AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.rh79, AAV.RHM4-1, AAV.hu37, AAVhu68, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and/or AAV.HSC16.

In some embodiments, the rAAV particles comprise a capsid protein of AAVhu68. In some embodiments, the rAAV particles comprise a capsid protein of AAV1. In some embodiments, the rAAV particles comprise a capsid protein of AAV9.

In some embodiments, the method further comprises the step of determining a yield of the purified rAAV particles. In some embodiments, the yield of the purified rAAV particles is between 65% and 99%.

In some embodiments, the method further comprises the step of determining enrichment of full rAAV particles in the purified rAAV particles. In some embodiments, at least 80% of the purified rAAV particles are full rAAV particles. In some embodiments, at least 85% of the purified rAAV particles are full rAAV particles. In some embodiments, 1% to 40% of the rAAV particles in the feed composition are full rAAV particles.

In some embodiments, the rAAV particles in the feed composition further comprises partially filled rAAV particles.

In some embodiments, the chromatography media is a pre-packed chromatographic monolithic column media. In some embodiments, the chromatography media is a rigid, high-flow agarose matrix modified with dextran surface extenders and a strong quaternary ammonium (Q) anion exchanger.

In some embodiments, the purified rAAV particles have at least 95% of the potency of the rAAV particles in the feed composition.

In some embodiments, the feed composition comprises Poloxamer 188.

In some embodiments, the method further comprises the preceding step of contacting a sample comprising rAAV particles with an affinity chromatography media, thereby providing the feed composition. In some embodiments, the method further comprises the preceding step of preparing the sample comprising rAAV particles by depth filtration, concentration or diafiltration. In some embodiments, the method further comprises the preceding step of preparing the sample comprising rAAV particles by depth filtration, concentration and diafiltration.

In another aspect, the present disclosure provides a population of rAAV particles prepared by the method of disclosed herein. In some embodiments, a population of rAAV particles comprise a capsid protein of an AAV selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.rh79, AAV.RHM4-1, AAV.hu37, AAVhu68, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and/or AAV.HSC16. In some embodiments, the population of rAAV particles comprise a capsid protein of an AAV selected from AAV1, AAV9, and AAVhu68.

In some embodiments, at least 70% of the rAAV particles in the population are full rAAV particles. In some embodiments, at least 80% of the rAAV particles in the population are full rAAV particles. In some embodiments, at least 85% or at least 90% of the rAAV particles in the population are full rAAV particles.

In yet another aspect, the present disclosure provides a pharmaceutical composition comprising: the population of rAAV disclosed herein and a pharmaceutically acceptable excipient.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIGS. 1A-1B provide 280 nM and 260 nM UV absorption (mAU) profiles of rAAV particles containing AAVhu68 capsids purified by AEX under the low loading volume (1 column volume (1CV)) condition (FIG. 1A) or high loading volume (4 column volume (4CV)) condition (FIG. 1B).

FIG. 2 provides 280 nM and 260 nM UV absorption (mAU) profiles of rAAV particles collected from anion exchange chromatography (AEX) purification performed at high (3.65E+14 GC/mL Resin) and low loading (2.44E+14 GC/mL Resin) conditions without the on-column holding period.

FIG. 3 provides 280 nM and 260 nM UV absorption (mAU) profiles of rAAV particles purified from a feed composition containing 20 mM BTP buffer at pH 8.8 and eluted at pH 8.8.

FIG. 4 provides 280 nM and 260 nM UV absorption (mAU) profiles of rAAV particles containing AAVhu68 capsids purified from a sample dilution without poloxamer. Exclusion of Poloxamer reduced yields and percentages of full AAVhu68 particles.

FIGS. 5A-5C provide 280 nM and 260 nM UV absorption (mAU) profiles of rAAV particles purified from a condition involving a short loading duration (0.85 hr) (FIG. 5A) or a long loading duration (14.57 hr for FIG. 5B and 20.54 hr for FIG. 5C). The short loading duration was achieved by decreasing the loading volume and the long loading duration was achieved by increasing the loading volume.

FIG. 6 provides 280 nM and 260 nM UV absorption (mAU) profiles of rAAV particles purified by a method involving the step of holding a feed composition in solution for 24 hours prior to loading onto the AEX media. The feed composition contains rAAV particles suspended in 20 mM BTP buffer at high pH (pH 10.2).

FIG. 7 provides 280 nM and 260 nM UV absorption (mAU) profiles of rAAV particles purified by a method involving the step of loading the feed composition onto a prepacked chromatography monolithic column media at low pH (pH 8.8) followed by the step of holding the composition on column (in the chromatography media) at high pH (pH 10.2) for 3 hours. The feed composition contains rAAV particles suspended in 20 mM BTP buffer at low pH (pH 8.8).

FIG. 8 provides 280 nM and 260 nM UV absorption (mAU) profiles of rAAV particles purified by a method involving the step of holding the feed composition at high pH (pH 10.2) in solution for 24 hours prior to loading onto the AEX media (“24 hr hold”) or the step of holding the feed composition at high pH (pH 10.2) on column (in the AEX media) for 24 hours after loading (“on column hold”). The feed composition contains rAAV particles suspended in 20 mM BTP buffer at high pH (pH 10.2).

FIG. 9A and FIG. 9B provide % full rAAV particles measured by SEC-MALS detector (y-axis) plotted as a function of the time-on-media (x-axis) at pH 10.2. While each molecule is not in contact with the media for this duration, it's an approximation of the first molecule in contact with the media until the start of elution. The exception is circled in FIG. 9B where loading occurred at low pH and a 3-hour hold was added. FIG. 9B is an enlarged view of a subset of data (from 0 to 400 on the x-axis) from FIG. 9A.

FIG. 10 provides 280 nM and 260 nM UV absorption (mAU) profiles of rAAV particles purified by AEX after loading the feed composition on column hold for 3 hours. Results are from a low loading ratio condition (3.83E+13).

FIG. 11 provides 280 nM and 260 nM UV absorption (mAU) profiles of rAAV particles purified by AEX after loading the feed composition at a high loading pH (pH 10.2) without the holding step. The feed composition contained rAAV particles suspended in 20 mM BTP buffer at high pH (pH 10.2).

FIG. 12 provides 280 nM and 260 nM UV absorption (mAU) profiles of rAAV particles purified by AEX after loading the feed composition at a low loading pH (pH 8.8) in glycine buffer, followed by holding on column (in the chromatography media) at a high pH (pH 10.2) for a duration of 3 hours. The feed composition contains rAAV particles suspended in 20 mM glycine buffer at high pH (pH 8.8).

FIG. 13A and FIG. 13B provides 280 nM and 260 nM UV absorption (mAU) profiles of rAAV particles purified by AEX after loading the feed composition at a low loading pH (pH 8.8), followed by holding on column (in the chromatography media) at a high pH (pH 10.2) for a duration of 24 hours. The feed composition in FIG. 13A (fresh) and FIG. 13B (cleaning-in-place (CIP) through storage) contain rAAV particles suspended in 20 mM glycine buffer at low pH (pH 8.8).

FIG. 14 provides 280 nM and 260 nM UV absorption (mAU) profiles of rAAV particles purified by AEX after loading the feed composition at pH 8.9, followed by holding on column (in the chromatography media) at a high pH (pH 10.2) for a duration of 3 hours. The loading ratio was 12.8E+13. The feed composition contains rAAV particles suspended in 20 mM glycine buffer at low pH (pH 8.9).

FIG. 15 provides 280 nM and 260 nM UV absorption (mAU) profiles of rAAV particles purified by AEX after loading the feed composition at a low loading pH (pH 8.8), followed by holding on column at a high pH (pH 10.2) for a duration for 3 hours. The feed composition contains rAAV particles suspended in 20 mM BTP buffer at low pH (pH 8.8).

### 5. DETAILED DESCRIPTION

### 5.1. Definitions

The term “recombinant adeno-associated virus particle” or “rAAV particle” as used herein refers to a nuclease-resistant particle (NRP) which comprises a AAV capsid. The AAV capsid can package therein a heterologous nucleic acid molecule comprising an AAV 5′ and/or 3′ inverted terminal repeat sequence. In some cases, the AAV capsid does not package a heterologous nucleic acid molecule, forming an empty rAAV capsid. The heterologous nucleic acid molecule can comprise an expression cassette including a coding sequence operably linked to expression control sequences. The coding sequence can encode a therapeutic protein. Alternatively, the expression cassette can include a sequence for gene editing, shRNA, miRNA, or other therapeutic function.

In many instances, rAAV particles are referred to as DNase resistant particle (DRP). However, in addition to this endonuclease (DNase), exonucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled. In case of full rAAV particle, the AAV capsid surround around the expression cassette which is designed to deliver a transgene to a host cell and protects these packaged genomic sequences from degradation (digestion) during nuclease incubation steps designed to remove contaminating nucleic acids which may be present from the production process.

The term a “full rAAV particle” as used herein refers to an rAAV particle comprising an AAV capsid encapsulating a heterologous nucleic acid molecule comprising an AAV 5′ and/or 3′ inverted terminal repeat sequence. The heterologous nucleic acid molecule is also referred to as a “vector genome”. The term “empty rAAV particle” as used herein refers to an rAAV particle which lacks such a heterologous nucleic acid molecule. The term “partially filled rAAV particle” as used herein refers to an rAAV viral particle which contains only a partially packaged nucleic acid molecule which is insufficient to achieve expression of the gene product. These empty or partially filled rAAV particles are non-functional to transfer the heterologous nucleic acid molecule (e.g., a gene of interest, a minigene) to a host cell.

A composition comprising rAAV particles can be analyzed by ultraviolet absorbance at about 260 nm and 280 nm. Because the nucleic acid content of the capsid has a profound influence on the A260 and A280 absorbance data, the resulting A260/A280 ratio in the plot of the absorbance data can be used to support the identification of full AAV particles and empty or partially filled AAV particles. Typically. full rAAV particles containing the nucleic acid content within the capsid have a higher detection of UV260 over 280 nm, while empty and partially filled particles have a higher detection of UV280 over UV260 nm. The A260/A280 ratio can be obtained by integrating the peak area of individual species in the UV absorption (mAU) plot at both wavelengths and dividing the A260 measurement by the A280 measurement.

A composition comprising rAAV particles can be analyzed and characterized using analytical ultra-centrifugation (AUC) or Multi-angle Static Light Scattering (SEC-MALS) as described in Brunham et al. Analytical Ultracentrifugation as an Approach to Characterize Recombinant Adeno-Associated Viral Vectors. Hum Gene Ther Methods. 2015 December; 26(6):228-42 and McIntosh et al, Comprehensive characterization and quantification of adeno associated vectors by size exclusion chromatography and multi angle light scattering. Sci Rep. 2021 Feb. 4; 11(1):3012, which are incorporated by reference in their entireties herein.

The term, “potency of rAAV particles” or “potency” as used herein refers to capability of the rAAV particles to transfer their vector genome into target cells. The potency can be measured by detecting gene expression from the viral genome transferred to the target cells. In some cases, the potency is measured by quantifying the vector genome transferred to the target cells. The potency can be measured in vitro or in vivo.

The term, “loading duration” or “loading duration of a feed composition” as used herein refers to the time that it takes to load a feed composition onto a chromatography media. The loading duration of a feed composition can be measured as the time between the initial contact of the feed composition with the chromatography media and the last application of the feed composition onto the chromatography media. The loading duration can change depending on the volume of the feed composition, the capacity of the chromatography media and the loading speed.

The term, “holding duration” or “hold duration”, as used herein refers to the duration of holding during which a composition is kept in a steady state. The holding is often performed under a static condition. But holding can be performed under a dynamic condition if any change applied to the composition is consistent and steady. For example, a “holding duration on column” can refer to the time a feed composition is maintained in contact with a column without application of any new sample or buffer onto the column, or with continuous and steady application of the same sample or buffer onto the column.

A “holding duration in solution” refers to the time a feed composition is maintained in a solution (e.g., a holding buffer) in the absence of a chromatography media, typically prior to loading the feed composition onto a chromatography media.

The term, “column volume” or “CV” as used herein refers to the volume inside of a packed column not occupied by the media. The volume can include both the interstitial volume (volume outside of the particles) and the media's own internal porosity (pore volume). The column volume can be used as a unit. For example, 50CV or 50 column volume indicates 50 times of the column volume.

The term “time-on-media” as used herein refers to the time between initial contact of rAAV particles with the chromatography media and elution of the rAAV particles from the chromatography media. While each rAAV particle is not on the chromatography media for this duration, it is determined by the time from the initial loading of the feed composition on the chromatography media until the start of elution.

The term “time-on-column” as used herein refers to the time between initial contact of rAAV particles with the chromatography column and elution of the rAAV particles from the chromatography column.

### 5.2. Method of Purifying rAAV Particles

The present disclosure provides a method of purifying a recombinant adeno-associated virus (rAAV) particles. The method effectively separates rAAV particles containing a heterologous nucleic acid molecule comprising an AAV 5′ and/or 3′ inverted terminal repeat sequence flanking a DNA sequence (full rAAV particles) from rAAV particles deficient of the heterologous nucleic acid molecule (empty rAAV particles) or containing a partial heterologous nucleic acid molecule DNA (partially filled rAAV particles). In preferred embodiments, the rAAV composition purified and recovered by the method disclosed herein includes full rAAV particle significantly enriched compared to the feed composition.

The method of purifying rAAV particles comprises subjecting a feed composition comprising rAAV particles to a chromatography media under a condition that allows binding of the rAAV particles to the chromatography media. The rAAV particles bound to the chromatography media are washed and eluted. To improve the performance, the rAAV particles bound to the chromatography media can be incubated in a holding buffer for at least 0.5 hr. before being eluted.

Accordingly, in some embodiments, the method comprises:
a) providing a feed composition comprising the rAAV particles, wherein the rAAV particles in the feed composition comprise empty, partially filled and full rAAV particles;
b) loading the feed composition onto a chromatography media under a condition that allows binding of the rAAV particles to the chromatography media over a loading duration;
c) optionally, holding the rAAV particles bound to the chromatography media within a holding buffer for a hold duration, wherein the hold duration is 0.5 hr or longer;
d) eluting the rAAV particles from the chromatography media; and
e) recovering purified rAAV particles, thereby enriching full rAAV particles.

In some embodiments, the method comprises the step of holding the rAAV particles bound to the chromatography media for a hold duration of at least 0.5 hr.

In some other embodiments, the method comprises:
a) providing a feed composition comprising the rAAV particles, wherein the rAAV particles in the feed composition comprise empty, partially filled and full rAAV particles;
b) contacting the feed composition with a chromatography media for a time-on-media under a condition that allows binding of the rAAV particles to the chromatography media, wherein the time-on-media is at least 0.5 hr;
c) eluting the rAAV particles from the chromatography media; and
d) recovering purified rAAV particles, thereby enriching full rAAV particles.

In some embodiments, the chromatography purification method is used in combination with rAAV production and purification methods known in the art. The additional steps for generation of rAAV particles can be referred to as an upstream or downstream process depending on the order of the step relative of the chromatography purification method disclosed herein. Various modifications to the upstream and downstream processes can be made.

### 5.2.1. Providing a Feed Composition

The feed composition is a composition comprising rAAV particles to be purified. The rAAV particles in the feed composition comprise empty, partially filled and full rAAV particles. The feed composition is loaded onto the chromatography media.

In some embodiments, the feed composition comprises a loading buffer compatible with the chromatography media. In some embodiments, the feed composition comprises a loading buffer compatible with the anion exchange chromatography media. In some embodiments, the feed composition comprises a loading buffer compatible with the affinity chromatography media.

In some embodiments, the loading buffer comprises bis-tris propane (BTP). In some embodiments, the loading buffer comprises 10-30 mM BTP. In some embodiments, the loading buffer comprises 10 mM BTP, 15 mM BTP, 20 mM BTP, 25 mM BTP, or 30 mM BTP. In some embodiments, the loading buffer comprises glycine. In some embodiments, the loading buffer comprises 10-30 mM glycine. In some embodiments, the loading buffer comprises 10 mM glycine. 15 mM glycine, 20 mM glycine, 25 mM glycine, or 30 mM glycine.

In some embodiments, the loading buffer further comprises Poloxamer 188. In some embodiments, the loading buffer is devoid of Poloxamer 188.

In some embodiments, the loading buffer further comprises NaCl. In some embodiments, the loading buffer further comprises 10-400 mM NaCl. In some embodiments, the loading buffer further comprises 10-200 mM NaCl. In some embodiments, the loading buffer further comprises 10-100 mM NaCl. In some embodiments, the loading buffer further comprises 10-50 mM NaCl.

In some embodiments, the loading buffer comprises 20 mM BTP and 10 mM NaCl at pH 10.2. In some embodiments, the loading buffer comprises 20 mM glycine and 10 mM NaCl at pH 10.2.

In some embodiments, the loading buffer provides a condition that allows binding of rAAV particles to chromatography media when applied to the chromatography media. In some embodiments, the feed composition has a pH between 9.5 and 10.5. In some embodiments, the feed composition has a pH between 10 and 10.5. In some embodiments, the feed composition has a pH about 10.2. In some embodiments, the feed composition has a pH between 7.0 and 9.5. In some embodiments, the feed composition has a pH between 7.5 and 9.5. In some embodiments, the feed composition has a pH between 8.0 and 9.5. In some embodiments, the feed composition has a pH between 8.0 and 8.9. In some embodiments, the feed composition has a pH between 8.2 and 8.8. In some embodiments, the feed composition has a pH 8.3, 8.4, 8.5, 8.6, 8.7 or 8.8.

In some embodiments, less than 50% of the rAAV particles in the feed composition are full. In some embodiments, less than 40% of the rAAV particles in the feed composition are full. In some embodiments, less than 30% of the rAAV particles in the feed composition are full. In some embodiments, less than 20% of the rAAV particles in the feed composition are full. In some embodiments, less than 10% of the rAAV particles in the feed composition are full.

In some embodiments, at least 45% of the rAAV particles in the feed composition are full. In some embodiments, at least 40% of the rAAV particles in the feed composition are full. In some embodiments, at least 30% of the rAAV particles in the feed composition are full. In some embodiments, at least 20% of the rAAV particles in the feed composition are full. In some embodiments, at least 10% of the rAAV particles in the feed composition are full. In some embodiments, at least 5% of the rAAV particles in the feed composition are full. In some embodiments, at least 1% of the rAAV particles in the feed composition are full.

In some embodiments, 1% to 40% of the rAAV particles in the feed composition are full. In some embodiments, 1% to 35% of the rAAV particles in the feed composition are full. In some embodiments, 1% to 30% of the rAAV particles in the feed composition are full. In some embodiments, 1% to 25% of the rAAV particles in the feed composition are full. In some embodiments, 1% to 20% of the rAAV particles in the feed composition are full.

### 5.2.1.1 Preparation of Feed Composition

The feed composition can be prepared using an upstream process known in the art. For example, the feed composition can be generated by a rAAV production process followed by concentration and/or purification process.

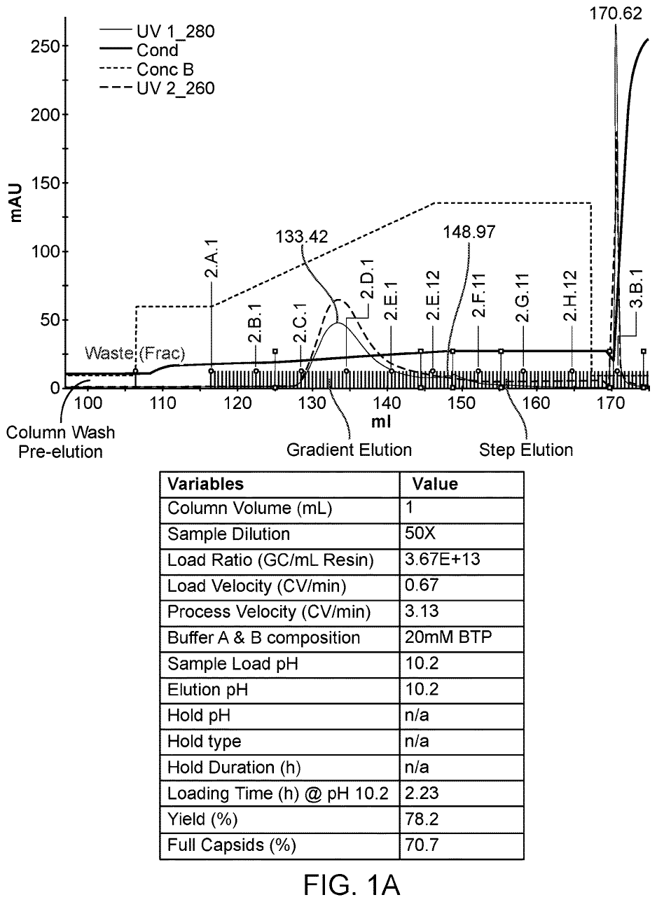

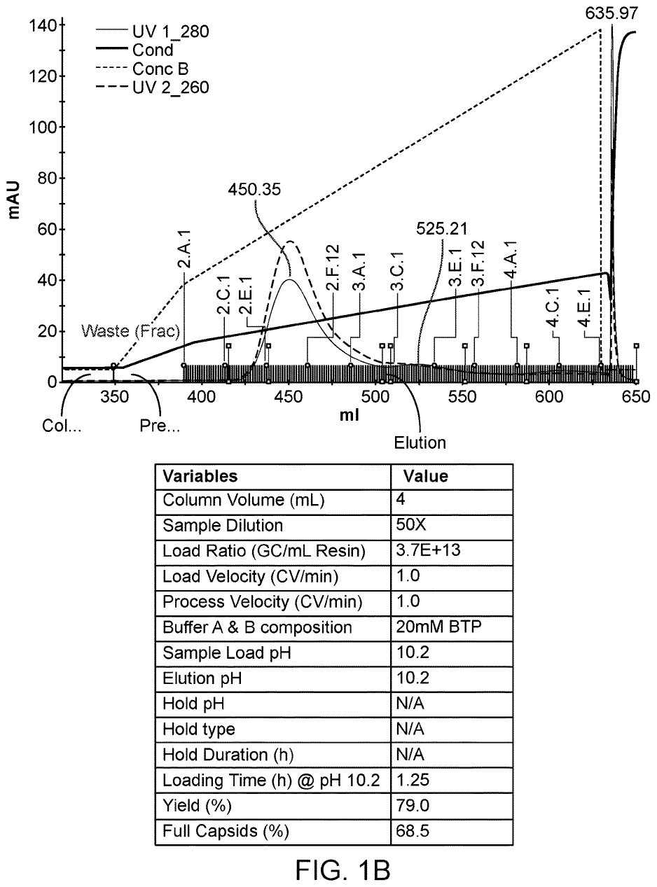

In some embodiments, the feed composition is prepared by a process involving (i) rAAV production, (ii) harvest treatment and lysis, and (iii) filtration. In some embodiments, the feed composition is prepared by a process involving (i) rAAV production, (ii) harvest treatment and lysis, and (iii) depth filtration and filtration. In some embodiments, the feed composition is prepared by a process involving (i) rAAV production, (ii) harvest treatment and lysis, (iii) depth filtration and filtration and (vi) TFF1 concentration and buffer exchange. In some embodiments, the process further includes affinity chromatography purification. In some embodiments, the feed composition is prepared by the process involving (i) cell bank thaw, (ii) inoculum expansion, (iii) rAAV production in a production bioreactor, (iv) harvest treatment and lysis, (v) depth filtration and filtration, (vi) TFF1 concentration and buffer exchange, and (vii) affinity chromatography. These steps can be used in different orders. Some exemplary processes are provided in FIG. 1.

5.2.1.1.1 rAAV Production

Numerous methods are known in the art for production of rAAV vectors including, but not limited to, production from a cell culture with transient transfection, stable cell line production, and infectious hybrid virus production systems which include Adenovirus-AAV hybrids, herpesvirus-AAV hybrids and baculovirus-AAV hybrids, as described in U.S. Pat. No. 11,098,286, which is incorporated herein in its entirety. rAAV production cultures for the production of rAAV virus particles generally require: 1) suitable host cells, including, for example, human-derived cell lines such as HeLa, A549, or 293 cells, or insect-derived cell lines such as SF-9, in the case of baculovirus production systems; 2) suitable helper virus function, provided by wild type or mutant adenovirus (such as temperature sensitive adenovirus), herpes virus, baculovirus, or a nucleic acid construct providing helper functions in trans or in cis; 3) functional AAV rep genes, functional cap genes and gene products; 4) a transgene (such as a therapeutic transgene) flanked by AAV ITR sequences; and 5) suitable media and media components to support rAAV production.

A variety of suitable cells and cell lines have been described for use in production of AAV. The cells can be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40. BMT 10, VERO, W138, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. In certain embodiments, the cells are suspension-adapted cells. In some embodiments, the cell line used for the rAAV particle purification method as described herein is an HEK 293 cell line.

The host cell can be a cell stably transformed with the sequences encoding rep and cap, and which is transfected with the adenovirus E1, E2a, and E40RF6 DNA and a construct carrying the expression cassette as described above. Other stable rep and/or cap expressing cell lines, such as B-50 as described (International Patent Application Publication No. WO 99/15685), or those described in U.S. Pat. No. 5,658,785, which are incorporated herein in their entirety, may be employed. Another desirable host cell contains the minimum adenoviral DNA which is sufficient to express E4 ORF6.

The required components for AAV production (e.g., adenovirus E1a, E1b, E2a, and/or E40RF6 gene products, rep or a fragment(s) thereof, cap, the expression cassette, as well as any other desired helper functions), may be delivered to the packaging host cell separately, or in combination, in the form of any genetic element which transfer the sequences carried thereon. Alternatively, one or more of the components required to be cultured in the host cell to package an expression cassette in an AAV capsid can be provided to the host cell in trans using a suitable genetic element.

Suitable media known in the art can be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors.

rAAV production culture media can be supplemented with serum or serum-derived recombinant proteins at a level of 0.5%-20% (v/v or w/v). Alternatively, as is known in the art, rAAV vectors can be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures can also include suspension-adapted host cells such as HeLa, 293, and SF-9 cells which can be cultured in a variety of ways including, for example, shake flasks, spinner flasks, stirred tank bioreactors, and disposable systems such as the Wave bioreactor system. In some embodiments, the host cells are cultured in a bioreactor, such as iCellis® bioreactor system (Pall). In some embodiments, host cells are cultured in a shake flask or a wave bioreactor for inoculum expansion before a large-scale culture. In some embodiments, host cells are cultured in a production bioreactor, such as iCellis® bioreactor system (Pall), after the inoculum expansion.

### 5.2.1.2 Harvest and Lysis

In some embodiments, the rAAV production culture is subjected to harvest and subsequent lysis of a production cell culture. rAAV vector particles of interest can be harvested from rAAV production cultures by lysis of the host cells of the production culture or by harvest of the media from the production culture, provided the cells are cultured under conditions known in the art to cause release of rAAV particles into the media from intact cells, as described more fully in U.S. Pat. No. 6,566,118, which is incorporated herein in its entirety. Suitable methods of lysing cells are also known in the art and include for example multiple freeze/thaw cycles, sonication, microfluidization, and treatment with chemicals, such as detergents and/or proteases.

At harvest, rAAV production cultures can contain one or more of the following in addition to rAAV particles: (1) host cell proteins; (2) host cell DNA; (3) plasmid DNA; (4) helper virus; (5) helper virus proteins; (6) helper virus DNA; and (7) media components including, for example, serum proteins, amino acids, transferrin and other low molecular weight proteins.

In some embodiments, the harvest is treated with a detergent at a slightly alkaline pH (e.g., 0.16% Lauryldimethylamine oxide (LDAO), 2 mM MgCl2, 20 mM Tris, 400 mM NaCl, pH 8.0). In some embodiments, the harvest is treated with a nuclease, or a combination of nucleases, to digest any contaminating high molecular weight nucleic acid present in the production culture. Such nucleases can target single stranded DNA, double-stranded DNA, or RNA. While the working examples illustrate use of a deoxyribonuclease (DNase) (e.g., Benzonase or Turbonuclease), other suitable nucleases are known, many of which are commercially available. The nuclease can be a DNAse, e.g., Benzonase® digestion performed under standard conditions known in the art. Thus, a suitable nuclease or a combination of nucleases, may be selected. Further, the nuclease(s) selected for this step may be the same or different from the nuclease(s) used during the upstream process or in the step more immediately following harvest of the cell culture. In some embodiments, the harvest is treated with a salt active nuclease (Salt Active Nuclease High Quality (SanHQ), 25U/mL).

5.2.1.3 rAAV Particle Clarification and Concentration

In some embodiments, the process of preparing a feed composition further includes tangential flow filtration (TFF) for concentrating the rAAV particles, heat inactivation of helper virus, rAAV capture by hydrophobic interaction chromatography, buffer exchange by size exclusion chromatography (SEC), and/or filtration. These steps can be used alone, in various combinations, or in different orders.

In some embodiments, the rAAV production culture harvest is clarified to remove host cell debris. In some embodiments, the production culture harvest is clarified by filtration through a series of depth filters including, for example, a grade DOHC Millipore Millistak+HC Pod Filter, a grade A1HC Millipore Millistak+HC Pod Filter, a 0.2 m Filter Opticap XL10 Millipore Express SHC Hydrophilic Membrane filter, and a Pall Supracap 50 depth filter capsule with dual-layer media grade PDK11 (2-20 μM retention rating) and luer-lock connections.

Clarification can also be achieved by a variety of other standard techniques known in the art, such as, centrifugation or filtration through any cellulose acetate filter of 0.2 μm or greater pore size known in the art. Still other suitable depth filters, e.g., in the range of about 0.045 μm to about 0.2 μm or other filtration techniques can be used. In some embodiments, clarification is performed by 0.2 μm filtration using Pall EKV 0.2 m filter. In some embodiments, the clarification step does not involve centrifugation. In some embodiments, the clarification is performed by ultrafiltration or diafiltration.

### 5.2.1.4 Tangential Flow Filtration (TFF) and Buffer Exchange

In some embodiments, the rAAV composition is concentrated via tangential flow filtration (“TFF”). Large scale concentration of viruses using TFF ultrafiltration has been described by R. Paul et al., HUMAN GENE THERAPY. 4:609-615 (1993). TFF concentration of the feed composition enables a technically manageable volume of feed composition to be subjected to the chromatography steps of the present invention and allows for more reasonable sizing of columns without the need for lengthy recirculation times. In some embodiments, the rAAV feed composition is concentrated between at least two-fold and at least ten-fold. In some embodiments, the feed composition is concentrated between at least ten-fold and at least twenty-fold. In some embodiments, the feed composition is concentrated between at least twenty-fold and at least fifty-fold. In some embodiments, the TFF step is performed using a membrane cassette (Pall Omega 100 kDa filters) to remove salt and protein. One of ordinary skill in the art will also recognize that TFF can also be used at any step in the purification process where it is desirable to exchange buffers before performing the next step in the purification process. In some embodiments, tangential flow filtration (TFF) is performed more than once, for example, both before and after chromatography purification.

5.2.2. Contacting a Feed Composition with a Chromatography Media

The method provided herein involves contacting a feed composition comprising rAAV particles with a chromatography media under a condition that allows binding of the rAAV particles to the chromatography media. The duration of the contacting step is referred to as a “time-on-media.” The time-on-media can be longer than 2 hrs. In some embodiments, the time-on-media is 2 to 24 hrs. In some embodiments, the time-on-media is longer than 3 hrs, 4 hrs, 5 hrs, 6 hrs, 7 hrs, 8 hrs, 9 hrs, 10 hrs, 1 hrs, or 12 hrs. In some embodiments, the time-on-media is shorter than 24 hrs, 22 hrs, 20 hrs, 18 hrs, 16 hrs, 14 hrs, or 12 hrs. In some embodiments, the time-on-media is less than 48 hrs, 36 hrs, or 24 hrs.

In some embodiments, the step of contacting comprises loading the rAAV particles onto the chromatography media over a loading duration. The loading duration can be longer than 0.5 hr, 1 hr, 2 hrs, 3 hrs, 4 hrs, 5 hrs, 6 hrs, 12 hrs, or 18 hrs. In some embodiments, the loading duration is 0.5 to 24 hours. The loading duration can be adjusted by changing the loading volume or loading rate.

In some embodiments, the step of contacting comprises holding the rAAV particles bound to the chromatography media within a holding buffer for a hold duration. In preferred embodiments, the hold duration is 0.5 hr or longer. In some embodiments, the holding buffer is identical to the loading buffer.

In some embodiments, the loading duration and the hold duration are 0.5 to 24 hrs in total. In some embodiments, the loading duration and the hold duration are at least 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, or 23 hrs in total.

In some embodiments, the step of contacting comprises washing the rAAV particles bound to the chromatography media with a washing buffer having pH 9.0 to 11. The washing can be performed after loading the feed composition onto the chromatography media but before holding the rAAV particles bound to the chromatography media. In some embodiments, the washing can be performed after holding the rAAV particles bound to the chromatography media. In some embodiments, the washing buffer can be identical to the loading buffer and/or the holding buffer.

5.2.3. Loading a Feed Composition with a Chromatography Media

The feed composition described above can be loaded onto a chromatography media for purification of rAAV particles and enrichment of full rAAV particles. After loading, the feed composition can contact the chromatography medium under a condition that allows binding of the rAAV particles to the chromatography media.

In some embodiments, the feed composition has been buffer exchanged with the column equilibration/loading buffer. In some embodiments, the feed composition is purified with an affinity chromatography media. In some embodiments, the feed composition is purified with an anion exchange chromatography (AEX). In some embodiments, the feed composition is purified with an affinity chromatography followed by an anion exchange chromatography. In some embodiments, the feed composition is purified with an anion exchange chromatography followed by an affinity chromatography.

In some embodiments, the process of preparing a feed composition for anion exchange chromatography comprises the step of affinity chromatography. In some embodiments, the process of preparing a feed composition for affinity chromatography comprises the step of anion exchange chromatography.

The chromatography media can have a load capacity at various ranges. In some embodiments, the load capacity is from 5E+10 to 5E+15 vg/mL of media. In some embodiments, the load capacity is from 5E+11 to 5E+15 vg/mL of media. In some embodiments, the load capacity is from 5E+11 to 5E+14 vg/mL of media. In some embodiments, the load capacity is from 5E+12 to 5E+14 vg/mL of media. In some embodiments, the load capacity is from 5E+13 to 10E+13 vg/mL of media.

In some embodiments, the loading takes at least 30 mins, at least 1 hr, at least 2 hr, at least 3 hrs, at least 4 hrs, at least 5 hrs, at least 6 hrs, at least 7 hrs, at least 8 hrs, at least 9 hrs, at least 10 hrs, at least 1 hrs, at least 12 hrs, at least 13 hrs, at least 14 hrs, at least 15 hrs, at least 16 hrs, at least 17 hrs. at least 18 hrs, at least 19 hrs, at least 20 hrs, at least 21 hrs, at least 22 hrs, at least 23 hrs, or at least 24 hrs.

In some embodiments, the loading takes less than 30 hrs, less than 24 hrs, less than 23 hrs, less than 22 hrs, less than 21 hrs, less than 20 hrs, less than 19 hrs, less than 18 hrs, less than 17 hrs, less than 16 hrs, less than 15 hrs, less than 14 hrs, less than 13 hrs, less than 12 hrs, less than 6 hrs, or less than 3 hrs.

In some embodiments, the loading takes 3-24 hrs, 6-24 hrs, 12-24 hrs, 18-24 hrs, or more than 24 hrs.

The loading time can be increased by increasing the loading volume or lowering the flow rate. The loading time can be decreased by decreasing the loading volume or raising the flow rate. In some embodiments, loading is performed as a continuous flow. In some embodiments, loading is performed as a discontinuous flow.

### 5.2.3.1 Affinity Chromatography

In some embodiments, the feed composition is loaded onto an affinity chromatography medium having binding specificity to rAAV. In some embodiments, the affinity chromatography is performed using an antibody-capture affinity chromatography medium. In some embodiments, affinity chromatography medium contains an rAAV-specific antibody, e.g., AAV1, AAV9, or AAVhu68 specific antibody, or other immunoglobulin construct specific to several rAAV serotypes.

In one embodiment, the chromatography medium comprises a solid support, which is a cross-linked poly(styrene-divinylbenzene) having an average particle size of about 50 m and having an rAAV-specific antibody. An example of such commercially available affinity resin is POROS™ high performance affinity resin (POROS CaptureSelect AAVX Affinity Resin) commercially available from Thermo Fischer Scientific. The resin contains ligands created by a technology based on camelid-derived single-domain antibody fragments coupled to the resin via carbonyldiimidazole (CDI). The ligand can comprise a single-domain fragment that comprises the 3 CDRs that form the antigen binding domain. In some embodiments, solid supports comprise polymeric matrix material, e.g., agarose, sepharose, sephadex, amongst others.

In some embodiments, the loading amounts is in the range of about 2×10¹² GC/mL to about 5×10¹⁴ GC/mL medium. In some embodiments, the loading amounts is in the range of about 2×10¹² GC/mL to about 5×10¹³ GC/mL medium. In some embodiments, the loading amounts is in the range of about 2×10¹² GC/mL to about 5×10¹² GC/mL medium.

In some embodiments, the maximum flow rate is between about 100 cm/hr to about 600 cm/hr (e.g., 350 cm/hr). In some embodiments, the maximum flow rate is between 100 cm/hr to 400 cm/hr. In some embodiments, the maximum flow rate is between 100 cm/hr to 300 cm/hr. In some embodiments, the maximum flow rate is between 100 cm/hr to 200 cm/hr. In some embodiments, the maximum flow rate is between 200 cm/hr to 400 cm/hr. In some embodiments, the maximum flow rate is between 300 cm/hr to 400 cm/hr. In some embodiments, the maximum flow rate is about 150 cm/hr. In some embodiments, the maximum flow rate is about 300 cm/hr. In some embodiments, the maximum flow rate is lower than 150 cm/hr. In some embodiments, the maximum flow rate is lower than 300 cm/hr.

In one embodiment, the feed composition containing the rAAV particles (including empty, partially filled and full particles) are loaded onto the chromatography medium in a buffer having a high salt concentration, e.g., about 400 nM NaCl to about 650 mM NaCl or other salt(s) having an equivalent ionic strength.

### 5.2.3.2 Anion Exchange Chromatography (AEX)

In some embodiments, the method provided herein comprises purification using anion exchange chromatography (AEX). Anion exchange chromatography (AEX) is a form of ion exchange chromatography that separates samples based on their net surface charge. Anion exchange chromatography (AEX) specifically uses positively charged ligands having affinity to targets having net negative surface charges.

The present invention relates to methods of purifying rAAV particles using various anion exchange chromatography (AEX) medium. Interactions between the AEX medium and rAAV particles are influenced by several factors, such as anion exchangers, flow rate, particle size of the resin, binding capacity, etc. The present invention further relates to specific conditions where rAAV particles can be effectively isolated, purified or sub-fractionated with the AEX medium. For example, specific buffer conditions for purification of rAAV particles with AEX are disclosed.

In some embodiments, the AEX medium comprises one or more amine functional groups. In some embodiments, the one or more amine functional group is selected from a primary amine, a secondary amine, a tertiary amine, a quaternary amine functional group, or combinations thereof. In some embodiments, the one or more amine functional group comprises a quaternary amine functional group. In some embodiments, the one or more amine functional groups are bound onto a resin, membrane, and/or nanofiber chromatographic media.

In some embodiments, the AEX medium comprises monolith. In some embodiments, the AEX medium is a monolith anion exchange medium. In some embodiments, the AEX medium is a monolith column. In some embodiments, the AEX medium is a pre-packed chromatographic monolithic column media. In some embodiments, the AEX medium is a CIMQA Monolith column (Sartorius). In some embodiments, the AEX medium comprise rigid, high-flow agarose matrix modified with dextran surface extenders and a strong quaternary ammonium (Q) anion exchanger.

Prior to loading, the AEX media can be equilibrated. In some embodiments, the AEX media is equilibrated with a loading buffer. In some embodiments the AEX media is equilibrated over multiple steps with different buffers.

In some embodiments, the feed composition has a high salt concentration when it is loaded onto the AEX column. In one embodiment, the feed composition has a salt concentration of about 400 mM NaCl to about 650 mM NaCl, or equivalent prior to being applied to the AEX medium. In one embodiment, the feed composition has a salt concentration of about 10 mM to about 300 mM NaCl. In one embodiment, the feed composition has a salt concentration of about 10 mM to about 200 mM NaCl. In one embodiment, the feed composition has a salt concentration of about 10 mM to about 100 mM NaCl. In one embodiment, the feed composition has a salt concentration of about 10 mM.

In some embodiments, the feed composition is in a loading buffer (Buffer A). In some embodiments, the buffer comprises 20 mM Bis-Tris-Propane (BTP). In some embodiments, the buffer comprises glycine. In some embodiments, the buffer comprises 10 mM, 15 mM, 20 mM, 25 mM, 30 mM or 50 mM Bis-Tris-Propane (BTP). In some embodiments, the buffer comprises 10 mM, 15 mM, 20 mM, 25 mM, 30 mM or 50 mM glycine. In some embodiments, the loading buffer comprises 20 mM BTP and 10 mM NaCl. In some embodiments, the loading buffer comprises 20 mM glycine and 10 mM NaCl.

In some embodiments, the feed composition has a pH between 9.5 and 10.5 before being applied to the AEX medium. In some embodiments, the feed composition has a pH between 10 and 11 before being applied to the AEX medium. In some feed embodiments, the composition has a pH of about 10.2 before being applied to the AEX medium. In some embodiments, the feed composition has a pH between 8 and 9 before being applied to the AEX medium. In some embodiments, the feed composition has a pH between 8.5 and 9.5 before being applied to the AEX medium. In some embodiments, the feed composition has a pH between 8.5 and 9 before being applied to the AEX medium. In some embodiments, the feed composition has a pH of about 8.8 before being applied to the AEX medium.

In some embodiments, the loading amounts is in the range of about 2×10¹² GC/mL to about 5×10¹⁴ GC/mL medium. In some embodiments, the loading amounts is in the range of about 2×10¹² GC/mL to about 5×10¹³ GC/mL medium. In some embodiments, the loading amounts is in the range of about 5×10¹² GC/mL to about 10×10¹³ GC/mL medium. In some embodiments, the loading amounts is in the range of about 1×10¹³ GC/mL to about 10×10¹³ GC/mL medium. In some embodiments, the loading amounts is in the range of about 1×10¹³ GC/mL to about 5×10¹³ GC/mL medium. In some embodiments, the loading amounts is about 3.0×10¹³ to 3.5×10¹³ GC/mL. In some embodiments, the loading amounts is about 2.0×10¹⁴ to 3.5×10¹⁴ GC/mL.

In some embodiments, the loading is performed at the loading velocity of 0.1-5.0 column volume (CV) mL/min AEX medium. In some embodiments, the loading is performed at the loading velocity of 0.5-3.0 CV mL/min AEX medium. In some embodiments, the loading is performed at the loading velocity of 0.5 CV-2.0 CV mL/min AEX medium. In some embodiments, the loading is performed at the loading velocity of 0.5 CV-1.5 CV mL/min AEX medium. In some embodiments, the loading is performed at the loading velocity of 0.5 CV-1.0 CV mL/min AEX medium. In some embodiments, the loading is performed at the loading velocity of about 0.5 CV, 0.6 CV, 0.7 CV or 0.8 CV mL/min AEX medium. In some embodiments, the loading is performed at the loading velocity of about 1.0 CV mL/min AEX medium. In some embodiments, the loading time is adjusted by changing the loading velocity or by changing the loading volume.

5.2.4. Washing rAAV Particles Bound to a Chromatography Media

In some embodiments, loading is followed by a washing step using a washing buffer. The washing step can improve purity or further aid in enriching, depleting, or isolating rAAV particles. The washing buffer can be a solution having specific pH ranges, salts, organic solvents, small molecules, detergents, zwitterions, amino acids, polymers, and any combination of the above. In some embodiments, the washing step is omitted. In the case, rAAV particles bound to the chromatography media are held within a holding buffer without being washed by a washing buffer. In some embodiments, the loading buffer is used for washing.

The washing step for the affinity chromatography can be performed with a washing buffer having a salt concentration higher than the loading buffer, e.g., in the range of about 750 mM to about 1 M NaCl or equivalent.

The washing step for the anion exchange chromatography can be performed with a washing buffer having a high pH. The washing buffer can have a pH between 9.0 and 11. In some embodiments, the washing buffer has a pH between 9.5 and 10.5. In some embodiments, the washing buffer has a pH of about 10.2.

In some embodiments, washing is performed at the washing velocity of 0.5-5 column volume/min chromatography medium. In some embodiments, washing is performed at the washing velocity of about 1 column volume/min chromatography medium. In some embodiments, washing is performed at the washing velocity of 1.0-10.0 column volume/min chromatography medium. In some embodiments, the washing is performed at the washing velocity of 1.0-5.0 column volume/min chromatography medium. In some embodiments, the washing is performed at the washing velocity of 2.0-4.0 column volume/min chromatography medium. In some embodiments, the washing is performed at the washing velocity of 3.0-4.0 column volume/min chromatography medium. In some embodiments, the washing is performed at the loading velocity of about 2.0, 2.5, 3.0, 3.5, 4.0 or 4.5 column volume/min chromatography medium.

In some embodiments, washing is performed with 0.5-50 column volume of the washing buffer. In some embodiments, washing is performed with 1-40 column volume of the washing buffer. In some embodiments, washing is performed with 1-25 column volume of the washing buffer. In some embodiments, washing is performed with 5-25 column volume of the washing buffer. In some embodiments, washing is performed with 10-20 column volume of the washing buffer.

In some embodiments, the washing step is performed with a washing buffer. In some embodiments, the washing buffer comprises 20 mM Bis-Tris-Propane (BTP). In some embodiments, the buffer comprises 10 mM, 15 mM, 20 mM, 25 mM, 30 mM or 50 mM Bis-Tris-Propane (BTP). In some embodiments, the washing buffer comprises 20 mM Bis-Tris-Propane (BTP) and NaCl. In some embodiments, the washing buffer comprises glycine. In some embodiments, the washing buffer comprises 10 mM, 15 mM, 20 mM, 25 mM, 30 mM or 50 mM glycine. In some embodiments, the washing buffer comprises 20 mM glycine and NaCl. In some embodiments, the washing buffer comprises 20 mM BTP and 10 mM NaCl at pH 10.2. In some embodiments, the washing buffer comprises 20 mM glycine and 10 mM NaCl at pH 10.2.

5.2.5. Holding rAAV Particles Bound to a Chromatography Medium

In some embodiments, the method of purifying rAAV particles comprises the step of holding rAAV particles bound to a chromatography medium in a holding buffer for a hold duration of at least 0.5 hr. The holding step can improve purify or further aid in enriching, depleting, or isolating full rAAV particles.

In some embodiments, the hold duration is 0.5 hr or longer. In some embodiments, the hold duration is at least 1 hr. In some embodiments, the hold duration is at least 2 hrs. In some embodiments, the hold duration is at least 3 hrs. In some embodiments, the hold duration is at least 6 hrs. In some embodiments, the hold duration is at least 12 hrs. In some embodiments, the hold duration is at least 15 hrs.

In some embodiments, the hold duration is less than 24 hrs. In some embodiments, the hold duration is less than 20 hrs. In some embodiments, the hold duration is less than 18 hrs. In some embodiments, the hold duration is less than 15 hrs. In some embodiments, the hold duration is less than 12 hrs. In some embodiments, the hold duration is less than 6 hrs. In some embodiments, the hold duration is less than 3 hrs. In some embodiments, the hold duration is not more than 3 hrs.

In some embodiments, the hold duration is 0.5 to 24 hrs. In some embodiments, the hold duration is 1 to 24 hrs. In some embodiments, the hold duration is 2 to 24 hrs. In some embodiments, the hold duration is 3 to 24 hrs. In some embodiments, the hold duration is 3 to 12 hrs. In some embodiments, the hold duration is 3 to 6 hrs. In some embodiments, the hold duration is 0.5 to 3 hrs. In some embodiments, the hold duration is about 3 to 12 hrs. In some embodiments, the hold duration is about 3 hrs. In some embodiments, the hold duration is about 12 hrs. In some embodiments, the hold duration is about 24 hrs. In some embodiments, the hold duration is less than 24 hrs.

In some embodiments, the holding buffer has pH 9.0 to 12. In some embodiments, the holding buffer has pH 9.0 to 11. In some embodiments, the holding buffer has pH 9.0 to 10.5. In some embodiments, the holding buffer has pH 9.5 to 10.5. In some embodiments, the holding buffer has a pH of about 10.2. In some embodiments, the holding buffer has a pH identical to the washing buffer. In some embodiments, the holding buffer is identical to the washing buffer. In some embodiments, the holding buffer is identical to the loading buffer. In some embodiments, the holding buffer comprises 20 mM BTP. In some embodiments, the holding buffer comprises 20 mM glycine.

In some embodiments, the holding is performed under a static condition without application of a sample onto the chromatography media. In some embodiments, the holding is performed under a dynamic condition with continuous application of a sample or buffer onto the chromatography media.

In some embodiments, when the loading duration of a feed composition is longer than 1 hr, the holding step is omitted. In some embodiments, when the loading duration of a feed composition is longer than 2 hrs, 3 hrs, 4 hrs, 5 hrs, 6 hrs, 7 hrs, 8 hrs, 9 hrs, 10 hrs, 1 hrs, or 12 hrs, the holding step is omitted. In some embodiments, the loading duration and the hold duration is at least 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, or 23 hrs in total. In some embodiments, the time-on-media of the feed composition is at least 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, or 23 hrs. In some embodiments, the time-on-media of the feed composition is less than 24 hrs, 23 hrs, 22 hrs, 21 hrs, 20 hrs, 19 hrs, 18 hrs, 17 hrs, 16 hrs, 15 hrs, 14 hrs, 13 hrs, or 12 hrs. In some embodiments, the time-on-media of the feed composition is between 1 hr and 24 hrs. In some embodiments, the time-on-media of the feed composition is between 2 hrs and 24 hrs. In some embodiments, the time-on-media of the feed composition is between 3 hrs and 24 hrs. In some embodiments, the time-on-media of the feed composition is between 6 hrs and 24 hrs. In some embodiments, the time-on-media of the feed composition is between 6 hrs and 12 hrs.

5.2.6. Eluting rAAV Particles

Selective elution can be achieved by changing salt, phosphate, or calcium concentrations. changing pH, altering temperature, adding organic modifiers, organic solvents, small molecules, detergents, zwitterions, amino acids, polymers, polyols (sucrose, glucose, trehalose, mannose, sorbitol, mannitol, glycerol, etc.), anti-oxidants (e.g.. methionine), EDTA, EGTA, Polysorbate 20, Polysorbate 80, ethylene glycol, propylene glycol, polyethylene glycol, polypropylene glycol, or urea, adding excipients that alter the surface tension of the solution, adding excipients that alter the polarity of the solution, altering the residence time to take advantage of differential desorption rates between full and empty rAAV particles, or any combination of the above.

Elution can be achieved with multiple elution buffers having different properties, such as pH, salts, organic solvents, small molecules, detergents, zwitterions, amino acids, polymers, temperature, and any combination of the above. A plurality of eluted fractions can be collected, wherein rAAV particles collected in each fraction have different properties. For example, rAAV particles collected in one fraction has a higher purity, a smaller or larger average size, a preferred composition, etc. than rAAV particles in other fractions. Such elution buffers with different properties can be applied as a continuous flow, while a plurality of eluted fractions are collected.

In some embodiments, the eluting step is performed with a linear salt gradient. In some embodiments, the linear salt gradient comprises between about 0.001 mM NaCl and about 1000 mM NaCl. In some embodiments, the linear salt gradient comprises between about 0.001 mM NaCl and about 100 mM NaCl. In some embodiments, the linear salt gradient comprises between about 0.01 mM NaCl and about 100 mM NaCl. In some embodiments, the linear salt gradient comprises between about 1 mM NaCl and about 100 mM NaCl.

In some embodiments, the eluting step is performed with a step salt gradient. In some embodiments, the step salt gradient comprises between about 7 mM NaCl and about 500 mM NaCl. In some embodiments, the step salt gradient comprises between about 70 mM NaCl and about 500 mM NaCl. In some embodiments, the step salt gradient comprises between about 70 mM NaCl and about 100 mM NaCl.

5.2.7. Recovering Purified rAAV Particles

The method of purifying rAAV particles comprises the step of recovering or collecting rAAV particles from the elution. The purified rAAV particles can be in an eluate from the elution. The eluate can be analyzed to determine purity of the purified rAAV particles (e.g., percentage [%] of full rAAV particles).

Accordingly, the method can further comprise the step of analyzing the purified rAAV particles. In some embodiments, the method comprises the step of determining a yield of the purified rAAV particles. In some embodiments, the method comprises the step of determining a yield of the full rAAV particles. The yield can be determined as a ratio between the total purified rAAV particles in the eluate and the total rAAV particles in the feed composition.

In some embodiments, the yield is at least 50%. In some embodiments, the yield is at least 60%, 70%, 80%, 90% or 95%. In some embodiments, the yield is between 65% and 99%. In some embodiments, the yield is between 70% and 99%. In some embodiments, the yield is between 75% and 99%. In some embodiments, the yield is between 80% and 99%. In some embodiments, the yield is between 80% and 95%.

In some embodiments, the method comprises the step of determining enrichment of full rAAV particles in the purified or recovered rAAV particles. In some embodiments, the method comprises the step of determining enrichment of full rAAV particles in the purified rAAV particles compared to the full rAAV particles in the feed composition. In some embodiments, the method comprises the step of determining the percentage (%) of full rAAV particles among the purified rAAV particles.

In some embodiments, at least 60% of the purified rAAV particles are full rAAV particles. In some embodiments, at least 65% of the purified rAAV particles are full rAAV particles. In some embodiments, at least 70% of the purified rAAV particles are full rAAV particles. In some embodiments, at least 75% of the purified rAAV particles are full rAAV particles. In some embodiments, at least 80% of the purified rAAV particles are full rAAV particles. In some embodiments, at least 85% of the purified rAAV particles are full rAAV particles. In some embodiments, at least 90% of the purified rAAV particles are full rAAV particles. In some embodiments, at least 95% of the purified rAAV particles are full rAAV particles.

In some embodiments, the purified rAAV particles have at least 99% of the potency of the rAAV particles in the feed composition. In some embodiments, the purified rAAV particles have at least 98% of the potency of the rAAV particles in the feed composition. In some embodiments, the purified rAAV particles have at least 95% of the potency of the rAAV particles in the feed composition. In some embodiments, the purified rAAV particles have at least 90% of the potency of the rAAV particles in the feed composition. In some embodiments, the purified rAAV particles have at least 85% of the potency of the rAAV particles in the feed composition. In some embodiments, the purified rAAV particles have at least 80% of the potency of the rAAV particles in the feed composition. In some embodiments, the purified rAAV particles have at least 75% of the potency of the rAAV particles in the feed composition.

### 5.3. A Population of Purified rAAV Particles

In another aspect, the present disclosure provides a population of rAAV particles purified using the method disclosed herein. In some embodiments, the population comprises enriched full rAAV particles.

In some embodiments, at least 95% of the population is full rAAV particles. In some embodiments, at least 90% of the population is full rAAV particles. In some embodiments, at least 85% of the population is full rAAV particles. In some embodiments, at least 80% of the population is full rAAV particles. In some embodiments, at least 75% of the population is full rAAV particles. In some embodiments, at least 70% of the population is full rAAV particles. In some embodiments, at least 99%, 98%, 97%, 98%, 96% or 95% of the population is full rAAV particles.

In some embodiments, less than 5% of the population is empty rAAV particles. In some embodiments, less than 10% of the population is empty rAAV particles. In some embodiments, less than 15% of the population is empty rAAV particles. In some embodiments, less than 20% of the population is empty rAAV particles. In some embodiments, less than 25% of the population is empty rAAV particles. In some embodiments, less than 30% of the population is empty rAAV particles. In some embodiments, less than 1%, 2%, 3%, 4%, or 5% of the population is empty rAAV particles.

In some embodiments, less than 5% of the population is partially filled rAAV particles. In some embodiments, less than 10% of the population is partially filled rAAV particles. In some embodiments, less than 15% of the population is partially filled rAAV particles. In some embodiments, less than 20% of the population is partially filled rAAV particles. In some embodiments, less than 25% of the population is partially filled rAAV particles. In some embodiments, less than 30% of the population is partially filled rAAV particles. In some embodiments, less than 1%, 2%, 3%, 4%, or 5% of the population is partially filled rAAV particles.

In some embodiments, rAAV particles in the population comprises a capsid protein of an AAV selected from: AAVhu68, AAV9, AAV1, AAV2, AAV3, AAV4, AAV5, AAV6, AAV7, AAV8, AAV10, AAV-11, AAV-12, AAV-13, AAV-14, AAV-15, AAV-16, AAV.rh8, AAV.rh10, AAV.rh20, AAV.rh39, AAV.Rh74, AAV.rh79, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and/or AAV.HSC16. In some embodiments, rAAV particles in the population comprises a capsid protein of an AAV selected from: AAV9, AAV1, and AAVhu68.

### 5.4. Pharmaceutical Compositions

In yet another aspect, the present disclosure provides a pharmaceutical composition comprising the rAAV particles prepared by the method disclosed herein. The pharmaceutical composition can comprise a population of the purified rAAV particles and a pharmaceutically acceptable excipient.

In some embodiments, the rAAV particles comprise a heterologous nucleic acid molecule with a coding sequence for a therapeutic protein. In some embodiments, the heterologous nucleic acid molecule comprises a sequence for gene editing, shRNA, miRNA, or other therapeutic function. In some embodiments, the heterologous nucleic acid molecule further comprises a regulatory sequence operably linked to the therapeutic gene.

The pharmaceutical composition can be formulated for the intravenous, intramuscular, subcutaneous, intrathecal, intracranial, intracerebroventricular, intradermal, rectal, oral, vaginal, intranasal, or inhaled administration. In some embodiments, the pharmaceutical composition is formulated for injection or infusion.

The pharmaceutical composition can be used to deliver the rAAV to a mammalian subject (e.g., human subject) in need thereof.

The pharmaceutical composition can be formulated using one or more carriers, excipients, stabilizers and adjuvants to, for example: (1) increase stability; (2) increase cell transfection or transduction; (3) permit the sustained or delayed release; (4) alter the biodistribution (e.g., target the rAAV particle to specific tissues or cell types); (5) increase the translation of encoded protein in vivo; and/or (6) alter the release profile of encoded protein in vivo.

Formulations of the pharmaceutical compositions provided herein can include, without limitation, saline, which may be formulated with a variety of buffering solutions (e.g., phosphate buffered saline), lactose, sucrose, calcium phosphate, gelatin, dextran, agar, pectin, water, lipidoids, liposomes, lipid nanoparticles, polymers, lipoplexes, core-shell nanoparticles, peptides, proteins, nanoparticle mimics and combinations thereof.

Formulations of the pharmaceutical compositions described herein can be prepared by any method known or hereafter developed in the art of pharmacology. In general, such preparatory methods include the step of associating the active ingredient (i.e., rAAV particles) with a carrier and/or one or more other accessory ingredients (e.g., excipients, stabilizers and adjuvants).

A pharmaceutical composition in accordance with the present disclosure can be prepared, packaged, and/or sold in bulk, as a single unit dose, and/or as a plurality of single unit doses. As used herein, a unit dose refers to a discrete amount of the pharmaceutical composition including a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient (i.e., rAAV particles), the pharmaceutically acceptable carrier, and/or any additional ingredients in a pharmaceutical composition in accordance with the present disclosure can vary, depending upon the identity, size, and/or condition of the subject being treated and further depending upon the route by which the composition is to be administered.

Various carriers, excipients, stabilizers and adjuvants for formulating pharmaceutical compositions and techniques for preparing the composition are known in the art (see Remington: The Science and Practice of Pharmacy, 22nd Revised Ed., Pharmaceutical Press, 2012; incorporated herein by reference in its entirety). The use of suitable conventional carriers, excipients, stabilizers and adjuvants is contemplated within the scope of the present disclosure.

### 6. EXAMPLES

### 6.1. Example 1: AEX Purification of rAAV Particles

rAAV particles were purified by a process involving anion exchange chromatography (AEX). Various purification conditions such as buffer compositions have been tested, and preferred conditions were selected. Table 1 provides two exemplary conditions (Process A and Process B) tested for purification of rAAV particles.

TABLE 1
Exemplary process and condition of anion-exchange chromatography

| Step | Process A | Process B |
|---|---|---|
| Cleaning in- | 9 Steps including buffers: | 4 Steps with removal of following buffers: |
| Place (CIP)/ | 50 mM Tris pH 7.5 | 50 mM Tris pH 7.5 |
| Equilibration | 20 mM BTP pH 10.2 | 20 mM BTP pH 10.2 |
| Steps | 20 mM BTP 1M NaCl pH 10.2 | 20 mM BTP 1M NaCl pH 10.2 |
| Buffer A & | A: 20 mM BTP, 10 mM NaCl pH 10.2 | A: 20 mM Glycine, 10 mM NaCl pH 10.2 |
| Buffer B | B: 20 mM BTP, 310 mM NaCl pH 10.2 | B: 20 mM Glycine, 310 mM NaCl pH 10.2 |
| Load | 2 step adjustment to pH 10.2 | 1 step adjustment to pH ~8.8 |
| Adjustment | 20 mM BTP, 0.01% P188 pH 10.2 | 20 mM BTP, 0.01% P188, pH 8.9 |
| | 0.2M Bis-Tris Propane, pH 10.2 | |
| Load | No Maximum Range Set | 8.5 E+13 vg/mL of monolith |
| Capacity | | |
| (DBC) | | |
| Wash Step | 20 mM BTP, 10 mM NaCl pH 10.2 for | 20 mM Glycine, 10 mM NaCl pH 10.2 |
| | 10 CVs at 1 CV/min | for 20 CVs at 1 CV/min |
| Hold Step | No hold | 3 hr hold |
| Elution | 20-80% Buffer B over 60 CVs | 20-50% Buffer B over 30 CVs |
| Gradient & | Start: >5 mAU (280 nm) and ≥1.0 | Start: ≥10 mS/cm, >5 mAU (280 nm) |
| Collection | A260/A280 | and ≥1.0 A260/A280 |
| | End: <5 mAU (280), ≤1.0 A260/280 | End: 50% Buffer B |
| Strip | 8 Steps with multiple buffers | 2 Steps with Reduced buffers |

As summarized in Table 1, in Process A, a feed composition containing a mixture of empty, partial, and full capsids partially purified by upstream processing (e.g., depth filtration/diafiltration, TFF concentration and affinity chromatography) was diluted 50-fold in a loading buffer containing Bis-Tris-propane (BTP) buffer (20 mM BTP, 10 mM NaCl. pH 10.2). The same buffer can be used to equilibrate an anion-exchange column. The process involved a 2-step load adjustment to pH 10.2. The feed composition was diluted into BTP buffer (20 mM) containing 0.01% poloxamer 188 at pH 10.2, followed by 0.2 M BTP at pH 10.2. In Process A there was no maximum range set for the load capacity (dynamic binding capacity, DBC). The feed composition was applied onto the equilibrated anion-exchange column (e.g., CIMmultus-QA™ column, CaptoQ column) at pH 10.2. The rAAV particles bound to the chromatography media was washed with a BTP washing buffer (20 mM BTP, 10 mM NaCl, pH 10.2) for 10 CVs at 1 CV/min. The bound rAAV particles was eluted with a linear salt gradient to BTP buffer B (20-80% buffer B over 60 CVs). UV absorbance was monitored at 260 and 280 nm. The fraction when the ratio of A260/A280 reaches an inflection point (≥1) was collected and then stripped with multiple buffers that involved eight steps.

Process B illustrates another purification conditions of anion-exchange chromatography for purifying rAAV particles. This is a simplified process with less steps and reduced use of buffer. The process involved a 1-step load adjustment, instead of 2-step, to pH 8.8 by adding BTP buffer (20 mM) containing 0.01% poloxamer 188 at pH 8.9. The load capacity (dynamic binding capacity, DBC) was limited to about 8.5 E+13 VG/mL of monolith. Similar to Process A, rAAV particles bound to the chromatography media in the anion-exchange column (e.g., CIMmultus-QA™ column were washed with a glycine washing buffer (20 mM glycine, 10 mM NaCl, pH 10.2) for 20 CVs at 1 CV/min, followed by a 3 hour hold on column at pH 10.2. The bound rAAV particles were eluted in a linear salt gradient using glycine buffer B (20-50% buffer B over 30 CVs. UV absorbance was monitored at 260 and 280 nm. The fraction when the ratio of A260/A280 reaches an inflection point (≥1) was collected and then stripped with reduced buffers that involved two steps.

Process B provides multiple advantageous features over the conventional method. For example, Process B significantly reduces the number of steps, decreases processing time, reduces buffer quantities and total number of buffers prepared during the cleaning-in-place (CIP) and equilibration (CIP/EQ) steps. It was noted that CIP/EQ steps do not impact processing.

Process B replaces BTP buffer with glycine buffer. The adjustment enhanced buffering capacity of glycine for pH control of gradient step (glycine pKa=9.7 vs BTP pKa=9.0).

Process A required a 2-step load adjustment at a high pH (pH 10.2). By contrast, Process B used 1 step load adjustment to a low pH (pH 8.8). Process B lowered the pH to 8.8 or 8.9 to limit the amount of time the product remains at high pH (e.g., pH 10.2).

Process A had no maximum range set for the loading capacity (dynamic binding capacity (DBC)), whereas Process B limited the loading capacity to about 8.5 E+13 vg/mL of monolith. Typically, DBC of Loading was assessed at pH ˜8.8. At 8.5E+13 vg/mL, a 10% breakthrough was observed. Loading at manufacturing scale was approximated to be 35% (3×200 m²-400 mL CIMQA) and 59% (1×200 m²-80 mL CIMQA) of DBC.

During the wash step, Process B replaces BTP with 20 mM Glycine, 10 mM NaCl pH 10.2 for 20 CVs at 1 CV/min followed by a 3 hour hold on column. The adjustment increased quantity of full capsids time-on-media at pH 10.2, which increased the yield of full capsid collected.

Process B lowered the range of buffer gradient from 20-80% Buffer B over 60 CVs to 20-50% Buffer B to over 30 CVs and additional start criteria including a ≥10 conductivity (mS/cm), >5 milli-absorbance unit (mAU) at 280 nm and ≥1.0 A260/A280 (infliction point). The adjustment reduced elution time and reduced time prior to neutralization adjustment (time at pH 10.2). End collection at 50% Buffer B provided simplicity for manufacturing. Additional start criteria (e.g., A260/A280 ratio) simplification can be used for potential removal and simplification for manufacturing.

During the striping step, Process A involved 8 steps with multiple buffers while Process B involved two steps with reduced buffers. The adjustments decreased processing time, buffer quantities and total number of buffers prepared.

### 6.2. Example 2: Conditions for Purification of rAAV Particles by Anion-Exchange Chromatography

Convective Interactive Media (CIM) QA Monolith (CIMmultus-QA™, Sartorius), is a strong anion-exchange monolith chromatography column containing cross-linked, porous poly-methacrylate material with defined channel size distribution having a diameter of more than 1,000 nm. The monolith chromatography column was used to separate rAAV particles. The rAAV particles processed through upstream processing (e.g., depth filtration/diafiltration, TFF concentration and capture chromatography) was diluted 50-fold into a Bis-Tris-propane (BTP) buffer A (20 mM BTP, 10 mM NaCl, pH 10.2), also referred to as “loading buffer,” to form a feed composition. The pH of the feed composition was adjusted to 10.2.

To determine the effect of loading volume on the percentage of full rAAV capsids collected, a control experiment was performed using a feed composition containing AAVhu68. The 280 nM and 260 nM UV absorption (mAU) profiles is shown in FIGS. 1A and 1B.

Approximately 1 mL (low volume) of feed composition containing AAVhu68 (including empty, partially filled and full capsids) was loaded onto the CIMmultus-QA™. The loading ratio was 3.67E+13. The loading velocity was 0.67 CV/min and the process velocity was 3.13 CV/min. The loading duration was about 2.23 hours at pH 10.2. As shown in FIG. 1A, these conditions produced about 78.2% of AAVhu68 particle yields and about 70.7% full AAVhu68 capsids.

The process was repeated with a total amount of 4 mL (high volume) of AAVhu68 particles. Loading ratio was 3.7E+13. Loading velocity was 1 CV/min and process velocity was 1 CV/min. The loading duration was about 1.25 hours at pH 10.2. These conditions provided an equivalent loading ratio but lower amount of time on the column compared to the 1 mL (low volume) loaded at a slower velocity (0.67 CV/min). FIG. 1B shows there were about 79% AAVhu68 particle yields and about 68.5% of full AAVhu68 capsids.

The data demonstrates that an increase of time-on-media slightly increases the percentage of yield of particles or percentage of full capsid collected (separate from empty and partially filled capsids).

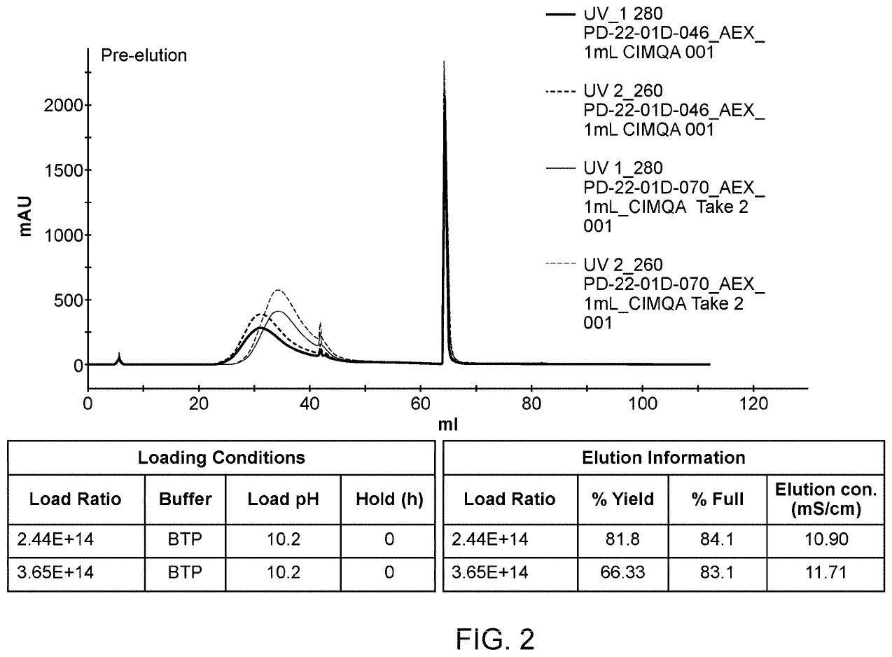

To determine the effect of high loading rate on the purification efficacy, the feed composition containing AAVhu68 particles (9E+13 Vg/mL, vector genome per milliliter) was loaded onto a 1 mL CIMmultus-QA™ column at a loading ratio (GC/mL resin) of 2.44E+14 or a loading ratio of 3.65E+14. Optionally, the column was pre-equilibrated with the loading buffer. The feed composition was allowed to flow through the column at a loading velocity rate of 0.67 mL/min. The column was washed in a washing buffer (20 mM BTP, 20 mM NaCl, pH 10.2), and then eluted with a salt gradient (20-50% gradient) in an elution buffer (20 mM BTP, 1M NaCl, pH 10.2). The volumetric flow rate was 1.54 CV/min. UV absorbance was monitored at 260 and 280 nm. The fraction when the ratio of A260/A280 reaches an inflection point (≥1) was collected and then stripped with buffer. The 280 nM and 260 nM UV absorption (mAU) profiles is shown in FIG. 2.

Referring to FIG. 2, the low loading ratio (2.44E+14) produced about 81.8% yields and about 84.1% of full rAAV capsids with an elution conductivity of about 10.9 ms/cm. The A260/A280 ratio was greater than one (1.26). In comparison, the high loading ratio (3.65E+14) produced about 66.33% yields and about 83.1% of full rAAV capsids with an elution conductivity of about 11.71 ms/cm. The A260/A280 ratio was also greater than one (1.37).

The data show that while high loading ratio at high pH (pH 10.2) decreases rAAV particle yields, it produces comparative percentage of full rAAV capsids collected. The slow flow rate in the low loading ratio experiment might have provided a longer time for the rAAV particles to flow through the column as the feed composition was being loaded dynamically.

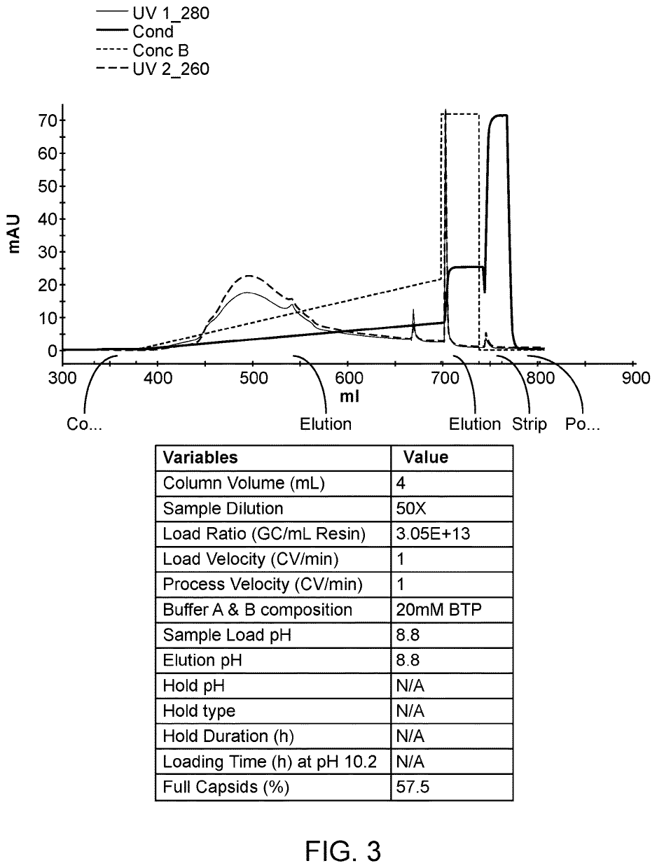

To determine the effect of loading pH on the yield and purity, a supernatant containing rAAV particles was diluted 50-fold in a loading buffer as described above, except magnesium chloride was omitted from the buffer (20 mM BTP, 10 mM NaCl, pH 10) to form a feed composition. The feed composition was adjusted to have a pH of 8.8. About 4 mL of the feed composition with a pH of 8.8 was loaded onto the CIMmultus-QA™ column at a normal loading rate of 3.05E+13. The loading velocity was about 1 CV/min and the process velocity was about 1 CV/min. The percentage of full rAAV capsids separated from empty and partially filled rAAV capsids was about 57.5%, as shown in FIG. 3.

The data indicates that loading at a low pH (e.g., pH 8.8) results in a lower percentage of full rAAV capsids, probably due to overlap of full and empty peaks (band broadening effect).

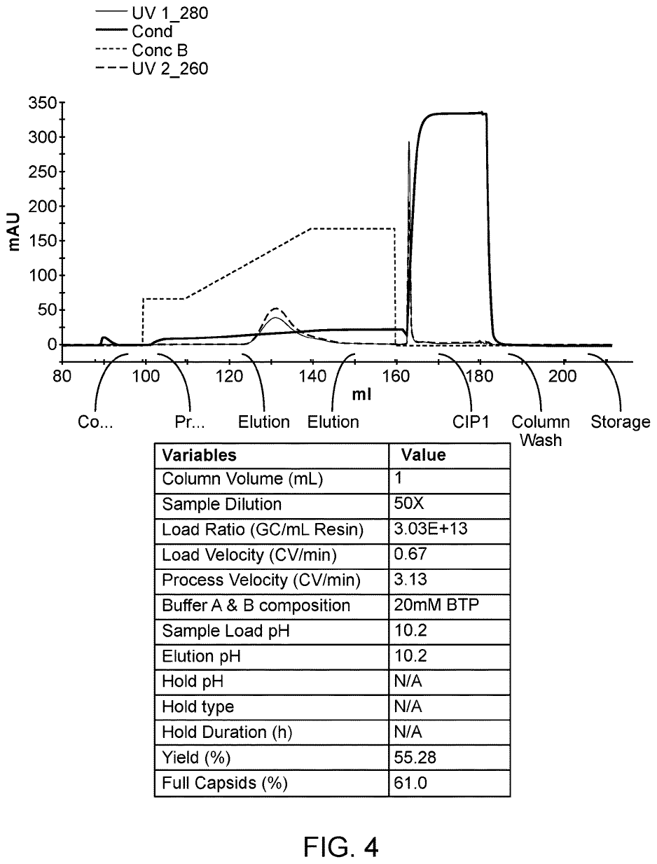

Poloxamer 188 (P188) is a nonionic linear copolymer having an average molecular weight of 8400 Daltons and is also referred to as PLURONIC F68, FLOCOR and RheothRx. Poloxamer 188 is generally used as a surfactant to stabilize rAAV particles. To determine whether the presence of poloxamer 188 impacts the purification of full rAAV capsids, a feed composition containing rAAV particles (e.g., AAVhu68) was prepared in a loading buffer (20 mM BTP, 10 mM NaCl, pH 10.2). No poloxamer 188 was added. The mixture was diluted 50-fold for loading. About 1 mL of feed composition with a pH of 10.2 was loaded onto the CIMmultus-QA™ column. The loading rate was 3.03E+13, loading velocity was about 0.67 CV/min, and process velocity was about 3.13 CV/min. The purification in the absence of poloxamer 188 resulted in a low yield of AAVhu68 particles (about 55.28%) and a low percentage of full rAAV capsids separated from empty and partially filled AAVhu68 capsids (about 61%), as shown in FIG. 4. The data suggests that poloxamer 188 aids in increasing yields and purity of rAAV particles.

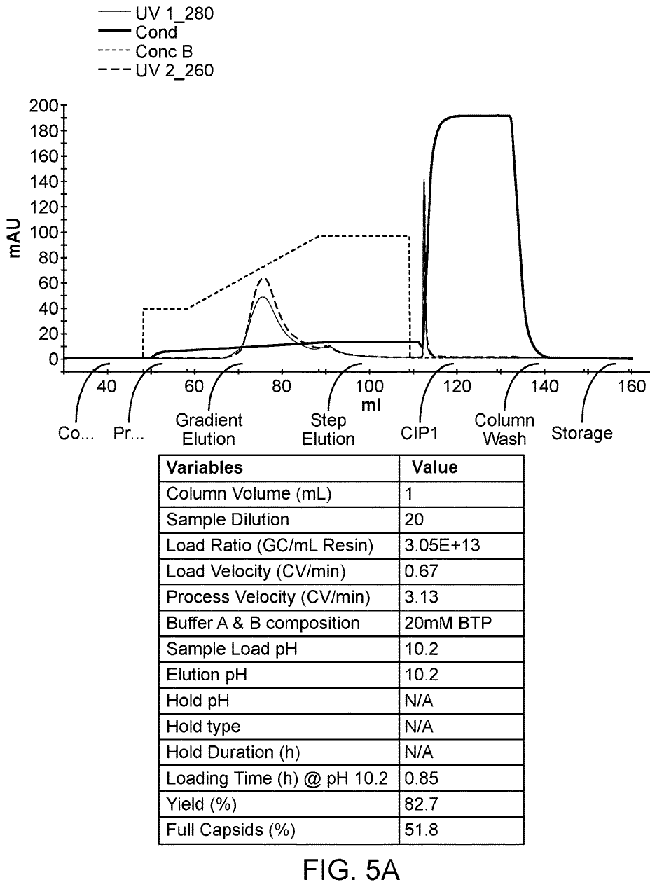

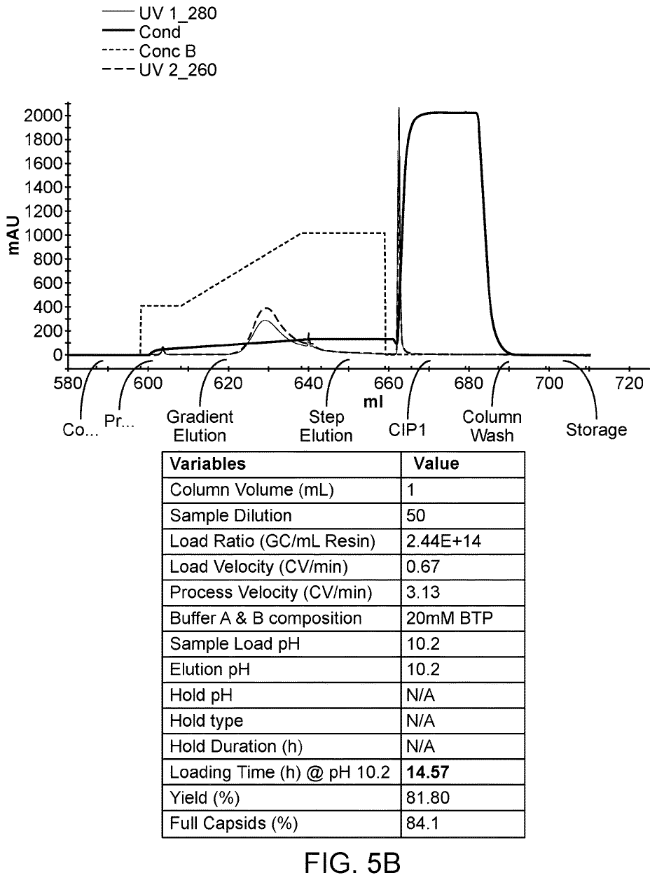

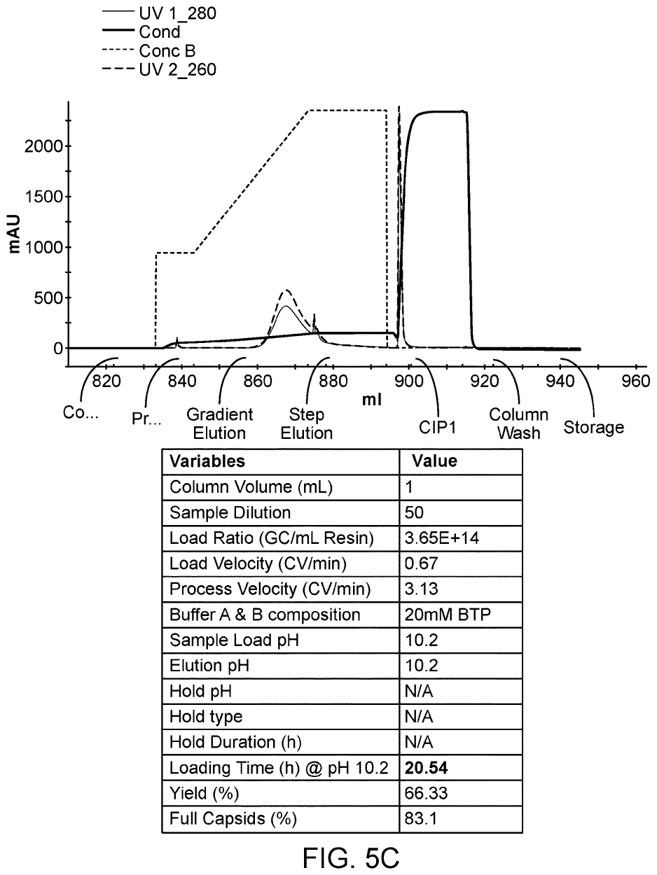

To determine effects of “Time-on-Media” on the rAAV particle purification, a feed composition was prepared in a loading buffer (20 mM BTP, 10 mM NaCl, pH 10.2). The mixture was diluted 20-fold prior to loading. About 1 mL (loading volume) of the feed composition was loaded onto the CIMmultus-QA™ column. The loading rate at 3.05E+13, loading velocity was about 0.67 CV/min, and process velocity was about 3.13 CV/min. The loading duration was about 0.85 hours at pH 10.2. Under these conditions, about 82.7% of rAAV particle yields were observed, but the percentage of full rAAV capsids separated from empty and partially filled rAAV capsids was about 51.8%, as shown in FIG. 5A.

In another experiment, 1 mL of a feed composition diluted 50-fold in a loading buffer (20 mM BTP, 10 mM NaCl, pH 10.2) was loaded onto the CIMmultus-QA™ column. The loading rate of 2.44E+14, loading velocity was about 0.67 CV/min, and process velocity was about 3.13 CV/min. In comparison to the 20-fold dilution (higher concentration), the loading duration of the 50-fold dilution loading feed composition was about 14.57 hours, which was increased by more than 12 hours. Notably, these conditions increased both the yield of rAAV particle (about 81.8%), and the purity of full rAAV capsids (about 84.1%). The 280 nM and 260 nM UV absorption (mAU) profiles is shown in FIG. 5B.

In another experiment, the loading ratio was increased to 3.65E+14, with the loading volume (1 mL) and load velocity (about 0.67 CV/min) and process velocity (about 3.13 CV/min) remained the same, the loading duration was about 20.54 hours. The data show higher loading ratio increases about 18 hours of loading duration when compared to the loading duration of a smaller loading volume as shown in FIG. 5A. FIG. 5C shows under such conditions, the yield of rAAV was about 66.33% and the percentage of full capsid was about 83.1%.

In this experiment, the time-on-media could be decreased by loading smaller volumes and the time-on-media could be increased by loading larger volumes (e.g., >12 hours longer time on the column as shown in FIG. 5B or >18 hours longer time on the column as shown in FIG. 5C). Overall, data from the experiment indicate that time-on-media is potentially a determining factor for improving full rAAV capsid separation. When the time-on-media increased, the yield and purity of full rAAV particles also increased.

### 6.3. Example 3: Holding on Media Improves Purification of rAAV Particles by Anion-Exchange Chromatography

rAAV particles were purified by AEX under a condition where the time-on-media was increased by holding the loaded sample on media for a hold duration.

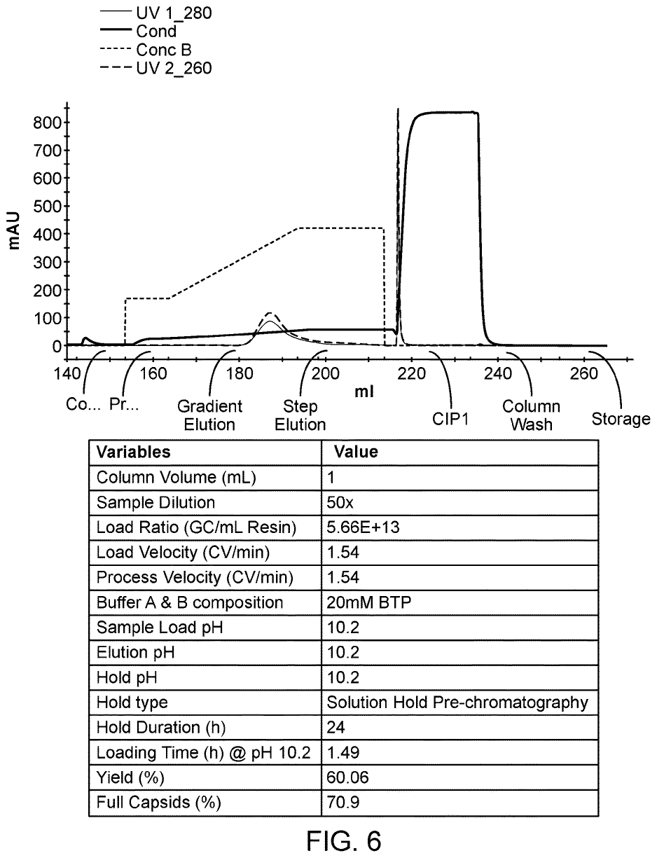

In a first experiment, a feed composition was prepared in a loading buffer (20 mM BTP, 10 mM NaCl, pH 10.2) and was diluted 50-fold. The feed composition was held in solution at pH 10.2 for a duration of 24 hours prior to loading onto the CIMmultus-QA™ column. After the holding period, 1 mL of the feed composition was loaded onto the column. The loading ratio was about 5.66E+13, loading velocity was about 1.54 CV/min, and process velocity was about 1.54 CV/min. The loading duration was about 1.49 hours. The column was washed with a washing buffer (20 mM BTP, 20 mM NaCl, pH 10.2) for 10 CVs at 1 CV/min, and eluted with a salt gradient (20-50% gradient) in an elution buffer (20-80% Buffer B, 20 mM BTP, 310 mM NaCl, pH 10.2, over 60 CVs). UV absorbance was monitored at 260 and 280 nm. The fraction when the ratio of A260/A280 reaches an inflection point (≥1) was collected and then stripped with buffer. The 280 nM and 260 nM UV absorption (mAU) profiles in FIG. 6 shows there were about 60.06% yield and about 70.9% full rAAV capsids. The data indicates that holding in solution at pH 10.2 does not increase purity of full capsids.

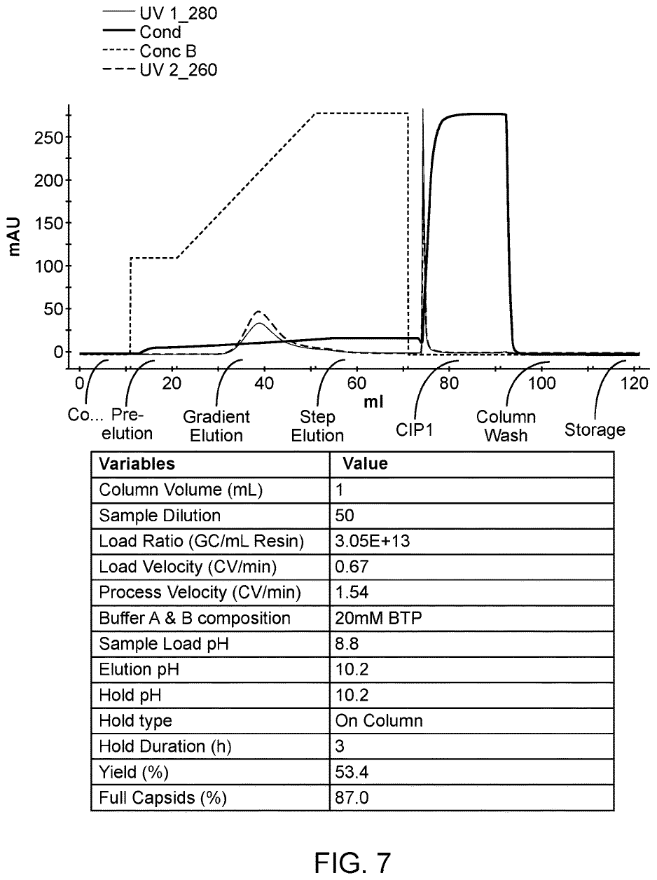

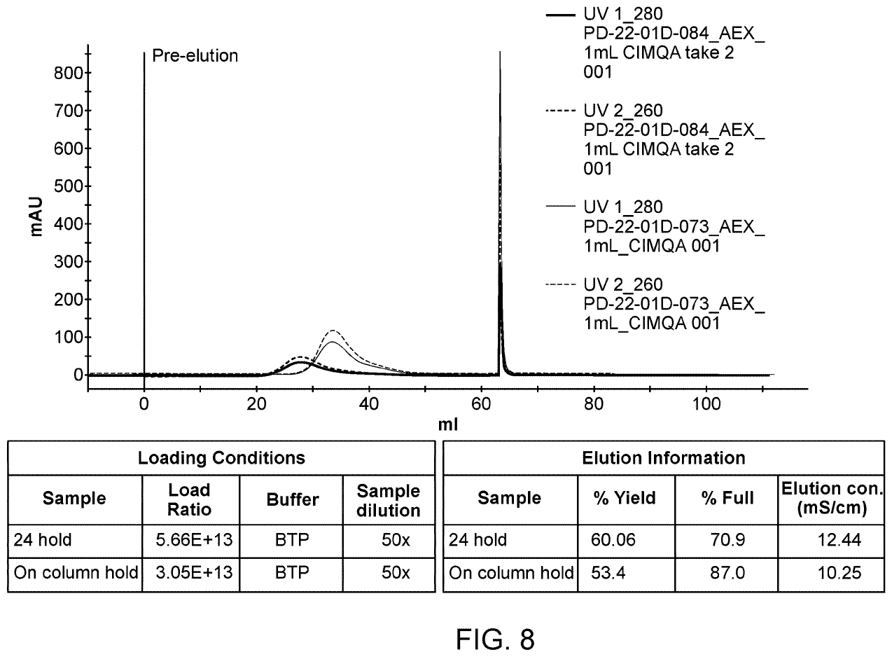

A second experiment was conducted under identical conditions except the holding step. The loading ratio was about 3.0E+13 VG/mL and the loading velocity was about 0.67 CV/min. After washing, the rAAV particles were allowed to hold at pH 10.2 (on column hold in contact with media) for a duration (e.g., time-on-media) of about 3 hours. As shown in FIG. 7, there were about 53.4% yield and about 87% full rAAV capsids. Comparing to in solution hold (about 70.9%, FIG. 6). on media hold (about 87%) significantly improved the percentage of full rAAV capsids by about 16.1%, as shown in a side-by-side comparison in FIG. 8. Additionally, comparing to loading and eluting at pH 8.8 (about 57.5%) as show in FIG. 4, loading at pH 8.8 and eluting at pH 10.2 (about 87%) drastically increases the percentage of full rAAV capsids by about 29.5%.

### 6.4. Example 4: Increased Time-On-Media Positively Correlates with Percentage of Full rAAV Capsid

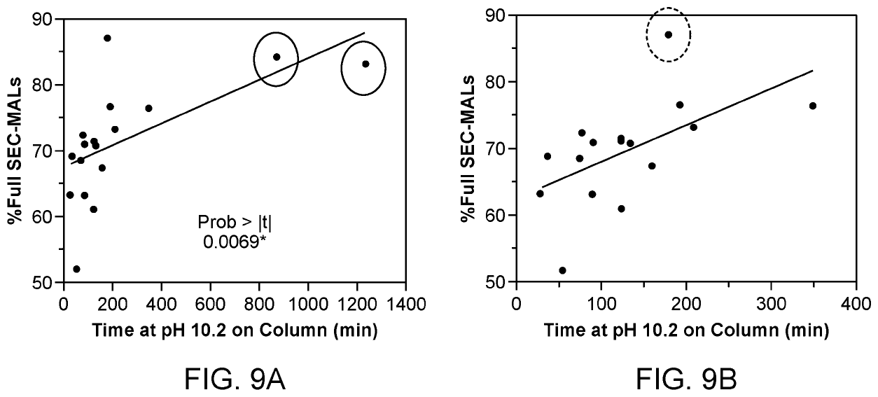

To determine the effect of time-on-media to purification efficacy, the experimental runs were plotted with the percentage of Size Exclusion Chromatography with Multiple-Angle Static Light Scattering (SEC-MALS) measurement (Y-axis) against time-on-media (X-axis), as shown in FIGS. 9A and 9B. All variable experiments with comparable conditions were included in the statistical analysis. “Time-on-Media” refers to loading duration at pH 10.2. While each molecule is not on the column in contact with the media for this duration, it's an approximation of the first molecule on the column in contact with the media until the start of elution. The exception is circled in FIG. 9B where loading occurred at low pH and a 3-hour hold was added. FIG. 9B is a subset of FIG. 9A and shows a time range between 0 to 400 minutes. FIG. 9B excludes high loading with long times on the column in contact with the media as the effect would not be linear at this point (circles in FIG. 9A). Experiment loaded at pH 8.8 and held on column in contact with the media (180 min) at pH 10.2 was included (circle in FIG. 9B).

The data show that decreasing flow rate increases time-on-media at pH 10.2. Similarly, increasing loading volume would increase time-on-media at pH 10.2. The effect is statistically significant (Prob>|t|=0.024, which is less than 0.05) for when including or excluding long duration of loading at pH 10.2 (increased time-on-media at 10.2). The data demonstrates that time-on-media at pH 10.2 positively correlated with percentage of full rAAV capsids purified and collected.

### 6.5. Example 5: Impact of Type of Buffer on Purification of rAAV Particles by Anion-Exchange Chromatography

To determine the effect of type of buffer on the percentage of full rAAV capsids collected, the feed composition was prepared in a loading buffer containing BTP (20 mM BTP, 10 mM NaCl, pH 10.2), or in a loading buffer containing glycine (20 mM Glycine, 10 mM NaCl, pH 10.2). Each of the mixture was diluted 50-fold prior to loading onto an 1 mL CIMmultus-QA™ column. The loading pH was adjusted to pH 8.8.

The feed composition in BTP loading buffer was loaded onto the column at a loading pH of 8.8, washed and eluted in BTP buffer at pH 10.2. The loading ratio was 3.05E+13. After washing, the rAAV particles were held on column in contact with the media (on column hold) at pH 10.2 for a duration of 3 hours. The results are shown in FIG. 7.

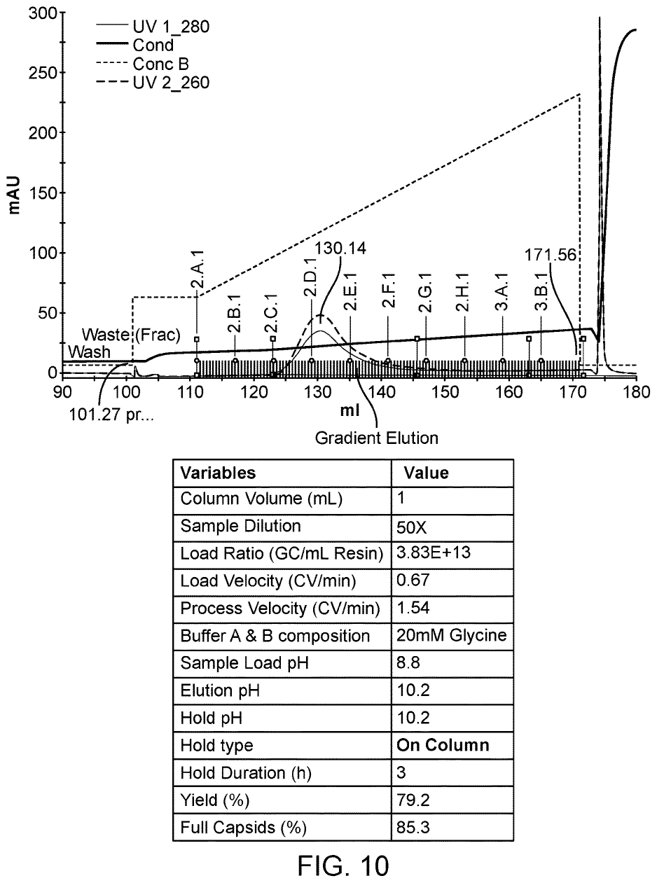

The feed composition in glycine buffer was loaded onto the column at pH 8.8. The loading ratio was 3.83E+13. The column was washed with a glycine washing buffer (20 mM glycine, 10 mM NaCl, pH 10.2) for 20 CVs at 1 CV/min, and followed by a 3 hour hold on column in contact with the media at pH 10.2. The bound rAAV particles were eluted in a linear salt gradient using glycine buffer B (20-50% buffer B over 30 CVs). UV absorbance is monitored at 260 and 280 nm. The fraction when the ratio of A260/A280 reaches an inflection point (≥1) was collected and then stripped with buffers. The results are shown in FIG. 10.

The 280 nM and 260 nM UV absorption (mAU) profiles shown in FIGS. 7 and 10 show that loading, washing, holding on column in contact with the media, and eluting in BTP buffer produces about 53.4% yields and about 87% full rAAV capsids. In comparison, loading, washing, holding on column in contact with the media, and eluting in glycine buffer produces about 79.2% yields and about 85.3% full rAAV capsids. The data suggests that percentage of full capsids is not impacted by buffer composition, but is by the hold on the column in contact with the media.

### 6.6. Example 6: Modification of Anion-Exchange Chromatography Process for Purification of rAAV Particles

This example illustrates modifications of the purification process for improving the efficiency of rAAV particle purification by anion-exchange chromatography.

The feed composition in glycine buffer was diluted 50-fold and loaded onto the column at pH 8.8. The loading ratio was about 3.83E+13 VG/mL and the loading velocity was about 0.67 CV/min. The column was washed with a glycine washing buffer (20 mM glycine, 10 mM NaCl, pH 10.2) for 20 CVs at 1 CV/min to wash off unbound components. The bound components (e.g., rAAV particles) were allowed to hold on column at pH 10.2 for a duration of 3 hour. The bound rAAV particles were eluted in a linear salt gradient using glycine buffer B (20-50% buffer B over 30 CVs) at pH 10.2. As shown in FIG. 10, the method produced about 79.2% yield and about 85.3% full rAAV capsid. The data indicate that decreasing sample loading pH to 8.8, washing with glycine buffer, and on column hold for 3 hours drastically increases the quantity of full rAAV capsid.

In comparison to the BTP buffer experiment, which produces about 87% full rAAV capsids (FIG. 7), use of glycine buffer (about 85.3%, FIG. 10) resulted in yields slightly lower percentage of full capsids although the difference was insignificant. The data suggest that the improvement of full rAAV capsids production is independent of BTP buffer. Additionally. glycine buffer has a higher pKa than BTP and offers increased pH control.

Overall, the data suggests that at pH 8.8 there is a significantly lower dynamic binding capacity as determined by the breakthrough at the front of the graph.

### 6.7. Example 7: Improved Purification of rAAV Particles by Anion-Exchange Chromatography is not AAV Serotype Dependent

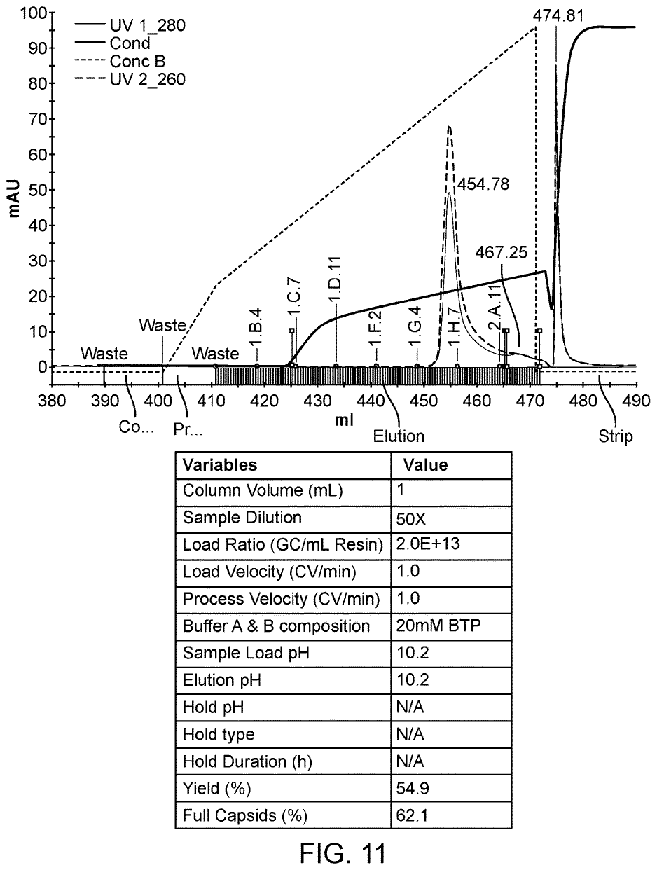

The rAAV particle purification procedures described in Example 6 were tested with a different AAV serotype, AAV1. The feed composition containing AAV1 was loaded onto a CIMmultus-QA™ column at a loading ratio of 2.0E+13 at pH 10.2. The column was washed and eluted with BTP buffer at pH 10.2. No holding was performed. FIG. 11 shows that the method produces 54.9% yields and 62.1% of full AAV1 capsids.

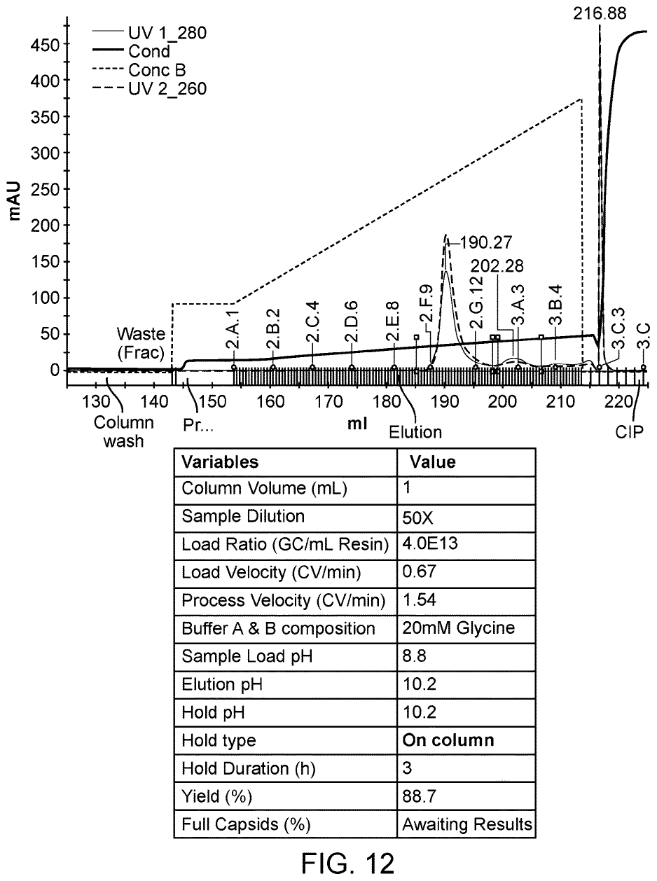

The feed composition containing AAV1 was loaded onto a CIMmultus-QA™ column at a loading ratio of 4.0E+13 at pH 8.8. The column was washed and eluted with glycine buffer at pH 10.2, and allowed to hold on column in contact with the media at pH 10.2 for about 3 hours. FIG. 12 shows the method produces 88.7% yields. The A260/A280 ratios were higher than control, indicating an increase in percentage of full AAV1 capsids.

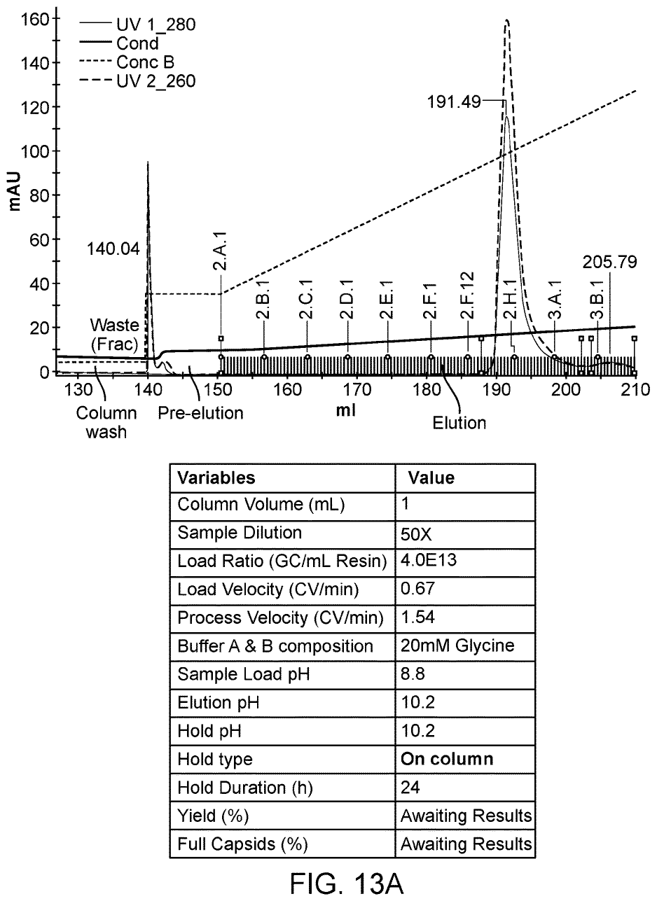

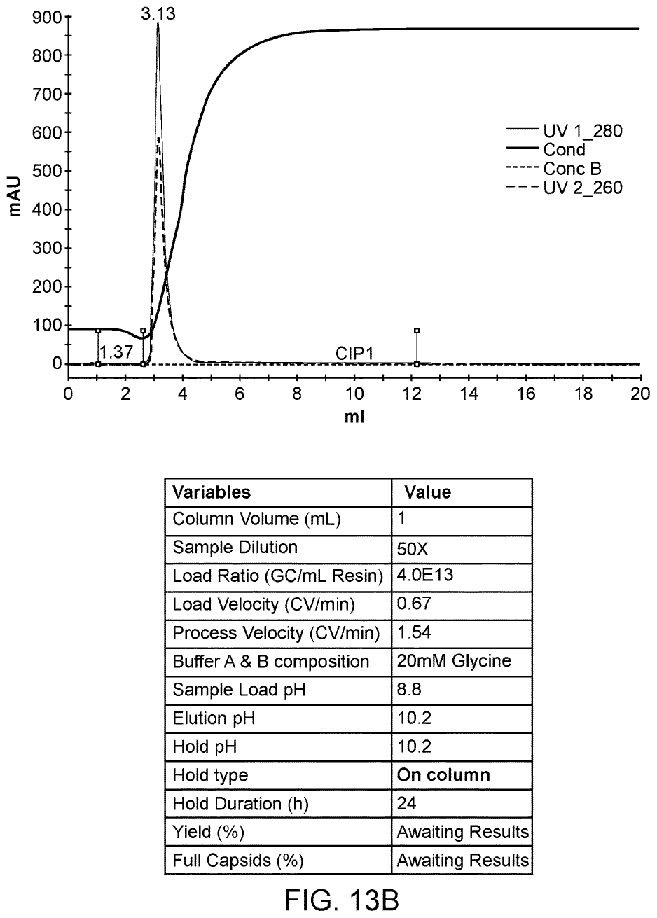

The third experiment was performed with extension of the holding time on column in contact with the media to 24 hours. The FPLC analysis results are shown in FIGS. 13A and 13B. The A260/A280 ratios were higher than control, indicating an increase in percentage of full AAV1 capsids.

The data demonstrate that modified rAAV particle purification process as described in Example 6 is applicable for multiple AAV serotypes.

### 6.8. Example 8: Scalable Anion-Exchange Chromatography Process for Purification of rAAV Particles

This example illustrates that the achievements of rAAV particle purification process as described in Examples 6 and 7 are scalable for manufacturing and production of high-quality full rAAV capsids in a large scale. The following experiments were performed to isolate AAV1 full capsids.

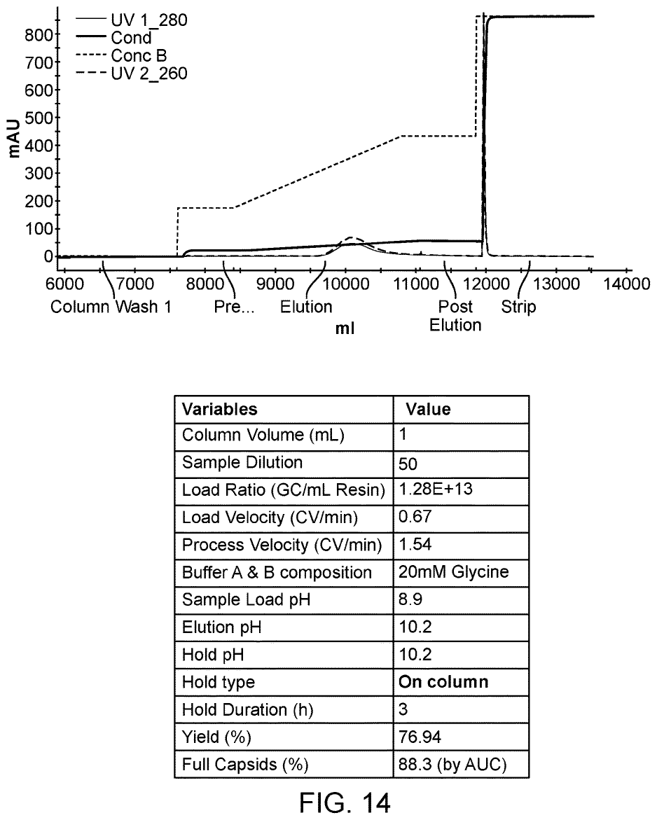

The experiment was performed with a slightly adjusted loading pH (pH 8.9 vs pH 8.8). The feed composition containing AAVhu68 was loaded onto a CIMmultus-QA™ column at a loading ratio of 1.28E+13 at pH 8.9. The column was washed and eluted with glycine buffer at pH 10.2. The rAAV particles bound to the chromatography media was allowed to hold on column at pH 10.2 for about 3 hours. The 280 nM and 260 nM UV absorption (mAU) profiles are shown in FIG. 14. The modified process achieved about 76.9% yield and about 88.3% (by AUC) of full AAVhu68 capsids. Notably, while a minimal trace of partial particles was normally detected in the full capsid pool collected using conventional methods, there was no detection of partial particles in the full capsid pool using the modified process.

### 6.9. Example 9: Application of an Improved Process in a Different Anion-Exchange Chromatography System for Purification of rAAV Particles

This experiment illustrates application of the modified processes described in Examples 6 and 7 using a different anion-exchange chromatography system for purification of rAAV particles.

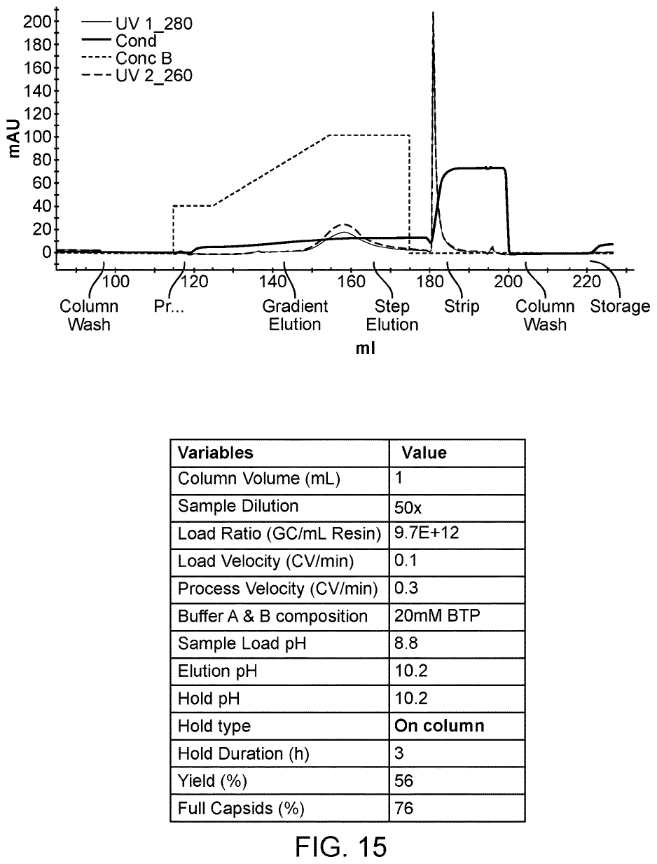

Capto Q Resin (Cytiva), is a strong anion-exchange resin chromatography column containing rigid, high flow agarose matrix modified with dextran surface extenders and quaternary ammonium (Q), was used to purify rAAV particles from upstreaming purification processing. The experiment was performed with adjustments to the loading pH. The feed composition was loaded at a loading ratio of 9.7E+12 at low pH (pH 8.8), washed with BTP buffer at pH 10.2 and held on column for about 3 hours at pH 10.2. The 280 nM and 260 nM UV absorption (mAU) profiles in FIG. 15 show that AAV1 particles likely bound more tightly to the chromatography media, which broadened the peak and potentially decreased yield. The method achieved about 56% yield and about 76% of full capsids. The data indicates that additional gradient run (e.g., extended gradient) may improve the production.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method of purifying a recombinant adeno-associated virus (rAAV) particles, the method comprising:
e) providing a feed composition comprising the rAAV particles, wherein the rAAV particles in the feed composition comprise empty and full rAAV particles;
f) contacting the feed composition with a chromatography media for a time-on-media under a condition that allows binding of the rAAV particles to the chromatography media, wherein the time-on-media is at least 0.5 hr;
g) eluting the rAAV particles from the chromatography media; and
h) recovering purified rAAV particles, thereby enriching full rAAV particles.

2. The method of claim 1, wherein, the time-on-media is longer than 2 hrs.

3. The method of claim 2, wherein the time-on-media is 2 to 24 hours.

4. The method of claim 2, wherein the time-on-media is longer than 3 hrs, 4 hrs, 5 hrs, 6 hrs, 7 hrs, 8 hrs, 9 hrs, 10 hrs, 11 hrs, or 12 hrs.

5. The method of any one of claims 1-4, wherein the step of contacting comprises holding the rAAV particles bound to the chromatography media within a holding buffer for a hold duration, wherein the hold duration is 0.5 hr or longer.

6. The method of claim 5, wherein the hold duration is 0.5 to 24 hours.

7. The method of claim 6, wherein the hold duration is not more than 3 hours.

8. The method of claim 7, wherein the hold duration is 0.5 to 3 hours.

9. The method of claim 8, wherein the hold duration is about 3 hours.

10. The method of any one of claims 5-9, wherein the holding buffer has a pH 9.0 to 11.

11. The method of claim 10, wherein the holding buffer has a pH of about pH 10.2.

12. The method of any one of claims 1-11, wherein the step of contacting comprises loading the rAAV particles onto the chromatography media over a loading duration.

13. The method of claim 12, wherein the loading duration is longer than 0.5 hr, 1 hr, 2 hrs, 3 hrs, 4 hrs, 5 hrs, 6 hrs, 12 hrs, or 18 hrs.

14. The method of claim 13, wherein the loading duration is 0.5 to 24 hours.

15. The method of any one of claims 12-14, wherein the loading duration and the hold duration are 0.5 to 24 hours in total.

16. The method of any one of claims 12-15, wherein the loading duration and the hold duration are at least 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, or 23 hrs in total.

17. The method of any one of claims 12-16, wherein the step of contacting comprises washing the rAAV particles bound to the chromatography media with a washing buffer having pH 9.0 to 11, after loading the feed composition onto the chromatography media but before holding the rAAV particles bound to the chromatography media.

18. The method of claim 17, wherein the washing buffer has a pH of between pH 9.5 to 10.5.

19. The method of claim 18, wherein the washing buffer has a pH of about pH 10.2.

20. The method of any one of claims 17-19, wherein the washing buffer comprises Bis-Tris propane (BTP) or glycine.

21. The method of any one of claims 1-20, wherein the feed composition has a pH between 8.0 and 8.9.

22. The method of any one of claims 1-21, wherein the chromatography media is an anion exchange chromatography media.

23. The method of any one of claims 1-22, wherein the chromatography media is an affinity chromatography media.

24. The method of any one of claims 1-23, wherein the eluting step is performed with a linear salt gradient.

25. The method of claim 24, wherein the linear salt gradient comprises between about 0.001 mM NaCl and about 1000 mM NaCl.

26. The method of claim 24, wherein the linear salt gradient comprises between about 0.001 mM NaCl and about 100 mM NaCl.

27. The method of any one of claims 1-23, wherein the eluting step is performed with a step salt gradient.

28. The method of claim 27, wherein the step salt gradient comprises between about 7 mM NaCl and about 500 mM NaCl.

29. The method of claim 27, wherein the step salt gradient comprises between about 70 mM NaCl and about 100 mM NaCl.

30. The method of any one of claims 12-28, wherein the loading step is performed by flowing feed composition through the chromatography media at a flow rate of between 0.1 CV/min and 5 CV/min.

31. The method of any one of claims 1-30, wherein the chromatography media comprises one or more amine functional groups.

32. The method of claim 31, wherein the one or more amine functional group is selected from a primary amine, a secondary amine, a tertiary amine, a quaternary amine functional group, or combinations thereof.

33. The method of claim 31, wherein the one or more amine functional group comprises a quaternary amine functional group.

34. The method of claims 31-33, wherein the one or more amine functional groups are bound onto a resin, membrane, and/or nanofiber chromatographic media.

35. The method of any one of claims 1-34, wherein the chromatography media comprises monolith.

36. The method of any one of claims 1-35, wherein the step of eluting is performed in a buffer having a pH between 9.5 and 10.5.

37. The method of any one of claims 1-36, wherein the rAAV particles comprise a capsid protein of an AAV selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.rh79, AAV.RHM4-1, AAV.hu37, AAVhu68, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and/or AAV.HSC16.

38. The method of claim 37, wherein the rAAV particles comprise a capsid protein of AAVhu68.

39. The method of claim 37, wherein the rAAV particles comprise a capsid protein of AAV1.

40. The method of claim 37, wherein the rAAV particles comprise a capsid protein of AAV9.

41. The method of any one of claims 1-40, further comprising the step of determining a yield of the purified rAAV particles.

42. The method of any one of claims 1-41, wherein the yield of the purified rAAV particles is between 65% and 99%.

43. The method of any one of claims 1-42, further comprising the step of determining enrichment of full rAAV particles in the purified rAAV particles.

44. The method of any one of claims 1-43, wherein at least 80% of the purified rAAV particles are full rAAV particles.

45. The method of claim 44, wherein at least 85% of the purified rAAV particles are full rAAV particles.

46. The method of any one of claims 1-45, wherein 1% to 40% of the rAAV particles in the feed composition are full rAAV particles.

47. The method of any one of claims 1-46, wherein the rAAV particles in the feed composition further comprises partially filled rAAV particles.

48. The method of any one of claims 1-47, wherein the chromatography media is a pre-packed chromatographic monolithic column media.

49. The method of any one of claims 1-47, wherein the chromatography media is a rigid, high-flow agarose matrix modified with dextran surface extenders and a strong quaternary ammonium (Q) anion exchanger.

50. The method of any one of claims 1-49, wherein the purified rAAV particles have at least 95% of the potency of the rAAV particles in the feed composition.

51. The method of any one of claims 1-50, wherein the feed composition comprises Poloxamer 188.

52. The method of any one of claims 1-51, further comprising the preceding step of contacting a sample comprising rAAV particles with an affinity chromatography media, thereby providing the feed composition.

53. The method of any one of claims 1-52, further comprising the preceding step of preparing the sample comprising rAAV particles by depth filtration, concentration or diafiltration.

54. The method of claim 53, further comprising the preceding step of preparing the sample comprising rAAV particles by depth filtration, concentration and diafiltration.

55. A population of rAAV particles prepared by the method of any one of claims 1-54.

56. The population of rAAV particles of claim 55, comprising a capsid protein of an AAV selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.rh79, AAV.RHM4-1, AAV.hu37, AAVhu68, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and/or AAV.HSC16.

57. The population of rAAV particles of claim 56, comprising a capsid protein of an AAV selected from AAV1, AAV9, and AAVhu68.

58. The population of rAAV particles of any one of claims 55-57, wherein at least 70% of the rAAV particles in the population are full rAAV particles.

59. The population of rAAV particles of claim 58, wherein at least 80% of the rAAV particles in the population are full rAAV particles.

60. The population of rAAV particles of claim 59, wherein at least 85% or at least 90% of the rAAV particles in the population are full rAAV particles.

61. A pharmaceutical composition comprising: the population of rAAV of any one of claims 55-60 and a pharmaceutically acceptable excipient.
